(19) United States
(12) Patent Application Publication
(10) Pub. No.: US 2026/0091131 A1
(43) Pub. Date: Apr. 2, 2026

(54) PRODUCTS AND METHODS FOR USE IN TREATING NDP-RELATED DISEASES

(71) Applicant: UCL BUSINESS LTD, London (GB)

(72) Inventors: Jane SOWDEN, London (GB); Valda PAUZUOLYTE, London (GB); James WAWRZYNSKI, London (GB); Aara PATEL, London (GB)

(21) Appl. No.: 19/120,184

(22) PCT Filed: Oct. 10, 2023
(86) PCT No.: PCT/GB2023/052621

§ 371 Date: Apr. 10, 2025

(30) Foreign Application Priority Data
Oct. 11, 2022 (GB) 2214972.8

(51) Int. Cl. A61K 48/00; A61K 9/00; A61K 38/00; A61P 27/12; A61P 27/16; C07K 14/475; C12N 15/86

(52) U.S. Cl. CPC A61K 48/005; A61K 9/0019; A61K 9/0048; A61K 48/0075; A61K 48/0083; A61P 27/12; A61P 27/16; C07K 14/475; C12N 15/86; A61K 38/00; C12N 2750/14143; C12N 2750/14145; C12N 2800/22

(57) ABSTRACT

The invention relates to a construct and AAV particle comprising a wildtype NDP nucleic acid sequence or a codon-optimised NDP nucleic acid sequence. The invention further relates to products and methods for the treatment of one or more of Norrie disease, age related hearing loss, diabetic maculopathy and retinopathy, retinal neovascularisation, retinal exudation, retinopathy of prematurity (ROP), Familial exudative vitreoretinopathy (FEVR), Coats disease, and other NDP-related diseases, or other beta catenin signalling-related retinal diseases.

### FIELD OF INVENTION

The present invention relates to a gene therapy construct expressing the NDP gene and an AAV particle for use in gene therapy. More particularly, the invention is a construct expressing a codon optimised NDP open reading frame for the treatment of NDP-related diseases.

### INTRODUCTION

Norrie disease (ND) is a rare recessive X-linked dual sensory disorder, caused by mutations in the NDP gene and manifesting as congenital blindness and progressive hearing loss (Fradkin 1971; Holmes 1971; Berger et al. 1992a; Berger et al. 1992b; Chen et al. 1992). Vision loss is caused by underdevelopment of the deep retinal vasculature, resulting in retinal detachment (Apple et al. 1974; Drenser et al. 2007). Hearing is usually normal in infants, but begins to gradually deteriorate from on average 12 years, often starting from a specific frequency region (Smith et al. 2012). A proportion of patients with Norrie disease have cognitive impairment and other neurological symptoms, or peripheral vascular disease and erectile dysfunction (Smith et al. 2012; Michaelides et al. 2004; Rehm et al. 1997; Cagao et al. 2018). No curative treatment exists for Norrie disease. However, the late onset of hearing loss provides an opportunity for early therapeutic intervention to preserve hearing.

NDP encodes norrin (norrin cystine knot growth factor (NDP)) a secreted soluble WNT analogue, which binds to a receptor complex, consisting of FZ-4, LRP-5/6 and TSPAN-12, to induce intracellular β-catenin signalling (Xu et al. 2004; Junge et al. 2009; Chang et al. 2015). This pathway is essential for the deep retinal vascularization and vascular barrier maintenance (Xu et al. 2004; Apple et al. 1974). Hearing loss in Norrie patients has been traced to the cochlea, and both vasculature and cochlear hair cells are affected (Nadol et al. 1990; Parving et al. 1978), consistent with norrin signalling being important for development or maintenance of these structures (Bryant et al. 2022; Hayashi et al. 2021; Rehm et al. 2002; Ye et al. 2011). The Ndp-KO mouse model recapitulates human Norrie disease (Nadol et al. 1990; Rehm et al. 2002; Berger et al. 1996). It has been demonstrated that these mice have an early cochlear vasculature morphology and barrier malformation, and reduction of endocochlear potential. This was followed by outer hair cell (OHC) degeneration in a “sensitive” tonotopic region and corresponding hearing loss in the mid-frequencies between 1 and 2 months of age (Bryant et al. 2022). This sequence of events implies that the outer hair cell degeneration is the tissue correlate of the auditory dysfunction, and the vascular pathology may be the primary cause of hearing loss in Norrie disease (Bryant et al. 2022).

Norrie disease is a good candidate for gene replacement therapy due to its small size (coding sequence of 402 bp) and simple structure (Ohlmann and Tamm 2012). Importantly, targeting NDP expression to a specific cell type may not be essential as norrin is secreted and there is evidence that it does not exert concentration gradient effects or provide directional cues (Wang et al. 2012; Ohlmann et al. 2005). Adeno-associated viral (AAV) vectors are favoured for clinical application due to their low immunogenicity and genotoxicity (Verdoodt et al. 2021). AAV9 has been shown to cross the blood brain barrier (Merkel et al. 2017) and transduce a broad range of cells, including in the retina and cochlea (Massaro et al. 2020; Shibata et al. 2017), and is already approved for use in clinic (Aslesh and Yokota 2022).

It is the object of the present invention to provide an early-stage treatment to preserve both cochlear and retinal structure and function. Treatment using the present invention achieves full to partial rescue of cochlear and ocular structure and function. This is the first treatment of NDP-related diseases, particularly Norrie disease, by AAV-mediated gene therapy at clinically relevant stages of disease progression.

### SUMMARY OF INVENTION

The invention relates to products and methods for the treatment of one or more of Norrie disease, age related hearing loss, diabetic maculopathy and retinopathy, retinal neovascularisation, retinal exudation, retinopathy of prematurity (ROP), Familial exudative vitreoretinopathy (FEVR), Coats disease, and other NDP-related diseases, or other beta catenin signalling-related retinal diseases.

According to a first aspect of the present invention, there is provided a construct comprising a wildtype NDP nucleic acid sequence or a codon-optimised NDP nucleic acid sequence.

Codon optimisation is advantageous because it is important to enable the viral dose to be reduced, which in turn reduces the risk of uveitis when injected into the vitreous and maybe also an immune response when injected into the cochlea.

In one embodiment, the wildtype NDP nucleic acid sequence or the codon-optimised NDP nucleic acid sequence is a human NDP open reading frame.

In one embodiment, the wildtype NDP nucleic acid sequence comprises the nucleotide sequence of SEQ ID NO: 1 or the codon-optimised NDP nucleic acid sequence comprises the nucleotide sequence of SEQ ID NO: 3.

In one embodiment, the NDP nucleic acid sequence comprises a nucleotide sequence having at least about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 or the NDP nucleic acid sequence comprises a nucleotide sequence having at least about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 3.

In one embodiment, the wildtype NDP nucleic acid sequence or the codon-optimised NDP nucleic acid sequence is under the control of a CAG promoter, a CBA promoter, CMV promoter, EF1a promoter, PGK promoter, TRE promoter, U6 promoter, UAS promoter, EFS promoter, SFFV promoter, MSCV promoter, SV40 promoter, UBC promoter, Pro1A promoter, hRHO promoter, hBEST1 promoter, Grm6 promoter, GJB2 promoter, a GJB6 promoter, a SLC26A4 promoter, a TECTA promoter, a DFNA5 promoter, a COCH promoter, a NDP promoter, a SYN1 promoter, a GFAP promoter, a PLP promoter, a TAK1 promoter, a SOX21 promoter, a SOX2 promoter, a FGFR3 promoter, a PROX1 promoter, a GLAST1 promoter, a LGR5 promoter, a HES1 promoter, a HES5 promoter, a NOTCH1 promoter, a JAG1 promoter, a CDKN1A promoter, a CDKN1B promoter, a SOX10 promoter, a P75 promoter, a CD44 promoter, a HEY2 promoter, a LFNG promoter, a S100b promoter, a CLDN11 promoter, an NDP promoter, or synthetic modifications or combinations of these promoters.

In one embodiment, downstream of the wildtype NDP nucleic acid sequence or the codon-optimised NDP nucleic acid sequence is a WPRE element or oPRE element. This is advantageous as it enhances expression and reduces immunogenic effects in the vitreous.

In one embodiment, the construct further comprises one or more of the following elements: 5′ and 3′ inverted terminal repeats, a self-cleaving P2A linker, a FLAG epitope sequence tagging C terminus, a simian virus 40 PolyA (SV40 late polyA) sequence, bovine growth hormone polyadenylation signal (BGHpA), an EGFP open reading frame sequence, and a pUC ori.

In one embodiment, the wildtype NDP nucleic acid sequence or the codon-optimised NDP nucleic acid sequence is incorporated into one or more of the following vectors: AAV1, AAV2, AAV3, AAV4, AAV5, AAV6, AAV7, AAV8, AAV9, AAV10, AAV-rh8, AAV-rh10, AAV-rh39, AAV-rh43, AAVAnc80, AAV 2/ShH10, AAV2/9, AAV-S vector, AAV-ie, or AAV-PHP.eB.

In one embodiment, the wildtype NDP nucleic acid sequence or the codon-optimised NDP nucleic acid sequence is incorporated into an AAV 2/ShH10 vector or AAV 2/9 vector or AAV-S vector.

In one embodiment, the construct is formulated for at least one of intravenous administration, intraocular administration, and/or intracochlear administration.

According to a second aspect of the invention, there is provided a construct comprising a wildtype NDP nucleic acid sequence or a codon-optimised NDP nucleic acid sequence according claims 1 to 10 for use in the treatment of one or more of Norrie disease, age related hearing loss, diabetic maculopathy and retinopathy, retinal neovascularisation, retinal exudation, retinopathy of prematurity (ROP), Familial exudative vitreoretinopathy (FEVR), Coats disease, and other NDP-related diseases, or other beta catenin signalling-related retinal diseases.

According to a third aspect of the invention, there is provided an AAV particle (NDP.AAV) comprising the construct of the first aspect of the invention.

According to a fourth aspect of the invention, there is provided an AAV particle according to the third aspect of the invention for use in the treatment of one or more of Norrie disease, age or diabetic related maculopathy and retinopathy, retinopathy of prematurity (ROP), Familial exudative vitreoretinopathy (FEVR), and other NDP-related diseases.

In one embodiment, the AAV particle is adapted to target cochlear fibrocytes, basal cells and marginal cells of the lateral wall spiral ligament (SL) and stria vascularis. and glial cells of the modiolus retinal Muller cells, Retinal Ganglion Cells, Retinal Pigment Epithelial cells and Photoreceptors, Retinal Ganglion Cells alone and/or Muller cells alone.

In one embodiment, the AAV particle according to the third aspect of the invention is administered intravenously.

In one embodiment, the AAV particle is an AAV 2/9 particle.

In one embodiment, the AAV particle according to the third aspect of the invention is administered intraocularly.

In one embodiment, the AAV particle is an AAV 2/ShH10 particle.

In one embodiment, the AAV particle according to the third aspect of the invention is administered intracochlearly.

In one embodiment, the AAV particle according to the third aspect of the invention the AAV particle is administered by at least one of intravenous administration, intraocular administration, and/or intracochlearly

In a further embodiment, NDP.AAV administration may be performed in any combination of intravenous administration, intraocular administration, and intracochlea administration. For example, administration of NDP.AAV may be by intravenous administration and intraocular administration or by intravenous administration and intracochlea administration or by intraocular administration and intracochlea administration or by intravenous administration, intraocular administration and intracochlea administration.

According to a fifth aspect of the invention, there is provided the ASV particle of the third aspect of the invention for use in treatment of one or more of Norrie disease, age related hearing loss, diabetic maculopathy and retinopathy, retinal neovascularisation, retinal exudation, retinopathy of prematurity (ROP), Familial exudative vitreoretinopathy (FEVR), Coats disease, and other NDP-related diseases, or other beta catenin signalling-related retinal diseases, wherein a dose is administered intraocularly neonatally or pre-natally, wherein, preferably, a dose is administered at about 14-17 postconceptual weeks (pcws), more preferably at about 15-16 pcws, wherein, alternatively, a dose is administered intraocularly, wherein, preferably, the dose is administered at about 22-26 postconceptual weeks (pcws), more preferably at about 24 pcws, wherein, alternatively, a dose is administered intraocularly at any time after birth.

According to a sixth aspect of the invention, there is provided the AAV particle according to the third aspect of the invention for use in treatment of one or more of Norrie disease, age related hearing loss, and other NDP-related diseases, wherein a dose is administered intracochlearly neonatally or pre-natally, wherein, preferably, a dose is administered at about 13-20 postconceptual weeks (pcws), more preferably at about 15-18 pcws, wherein, optionally, a dose is administered intracochlearly to an adolescent, preferably at about 12 years old and below, wherein, optionally, a dose is administered intracochlearly in adulthood, preferably ages 12 years and above.

According to a seventh aspect of the invention, there is provided the AAV particle according to the third aspect of the invention for use in treatment of one or more of Norrie disease, age or diabetic related maculopathy and retinopathy, retinopathy of prematurity (ROP), Familial exudative vitreoretinopathy (FEVR), and other NDP-related diseases, wherein a dose is administered intravenously, wherein the dose is administered pre-natally, or post natally, neonatally, in childhood or in adulthood.

According to an eighth aspect of the invention, there is provided method of rescuing vascular architecture in the ear and the eye and/or vascular barrier function in the ear and the eye, and or preventing or treating neovascularisation in the eye the method comprising the step of administering the NDP.AAV particle by at least one of intravenous administration, intraocular administration, and intracochlear administration at an early stage of development, preferably a pre-natal stage of development, or alternatively in the first year of life, preferably in the neonatal period. Treatment in the early stages of development to rescue vascular architecture and blood retinal barrier in ear & eye. In the ear this prevents death of sensory hair cells and progressive hearing loss. Treatment of neonatal stage mouse equivalent to prenatal human corrects the vascular architecture preventing of neovascularisation or restoration of normal vascular anatomy.

According to a ninth aspect of the invention, there is provided a method of rescuing the blood retinal barrier and/or barrier in the cochlear and/or the endocochlear potential, the method comprising the step of administering the NDP.AAV particle by at least one of intravenous administration, intraocular administration, and intracochlear administration at a late stage of development, preferably a post-natal stage of development. The present invention has been shown to prevent death of sensory hair cells and progressive hearing loss even when administered after development is complete. This means that treatment in life will be feasible. Treatment of juvenile/young adult mice ameliorates progressive hearing loss and retinal vessel leakiness. Barrier rescue involves tight junction and transcellular permeability recovery and normalisation of pericyte arrangement on vessels. With vessel barrier rescue in the cochlear and endocochlear potential rescue, sensory hair cells and hearing are protected without rescuing vascular morphology. The present invention therefore achieves long term amelioration of hearing loss (data at 15 months efficacy, i.e. mice in old age). The present invention when applied at a late-stage reduces vessel leakiness in retina prevents exudation, vision loss, secondary glaucoma, and loss of the eye as a result of phthisis. Therefore, here are significant advantages to administering NDP.AAV to post-natal and older individuals and it is indeed a surprising finding that retinal vessel integrity can be rescued after transduction of juvenile tissues and after retinal vessel development is complete because it indicates that hair cell rescue can be achieved independently of vascular architecture pathology.

For all aspects, sequence identity is determined by comparing two aligned substantially complementary sequences over their length and overall identity is expressed as a percentage. The measurement of nucleotide sequence identity is well known in the art, using specialist computer programs such as “BLAST”.

The nucleic acid sequence may be a DNA, RNA, cDNA, or PNA and may be recombinant or synthetic. It may be single stranded or double stranded.

The nucleic acid sequence may be derived by cloning, for example using standard molecular cloning techniques including restriction digestion, ligation, gel electrophoresis (for example as described in Sambrook et al; Molecular Cloning: A laboratory manual, Cold Spring Harbour laboratory Press). The nucleic acid sequence may be isolated or amplified using PCR technology. Such technology may employ primers based upon the sequence of the nucleic acid sequence to be amplified. With the sequence information provided, the skilled person can use available cloning techniques to produce a nucleic acid sequence or vector suitable for transduction into a cell.

### FIGURES

The invention is further described in the following non-limiting figures.

FIG. 1: Gene therapy construct evaluation and study design.

A. Schematic of the AAV expression construct. Expression is driven by ubiquitous CAG promoter and EGFP tags all transduced cells. Human NDP cDNA, containing its native secretion signal and a C-terminal flag tag, is connected via a P2A linker, which self-cleaves from the same transcript during translation and the tagged norrin is secreted from the cells.

(B-B″) Expression of construct in HEK293 cells. Intrinsic EGFP mostly colocalizes with anti-flag immunostaining (yellow). Scale bar: 50 μm.

C. Detection of GFP and NDP monomer bands in transfected HEK293 cell lysate. Extended view figure is available for this data.

D. Schematic of the β-catenin signalling and TopFlash assay. NDP dimer binds to FZ4 complex with essential co-receptor LRP5 or LRP6 and signal amplifying co-receptor TSPAN12, inducing β-catenin binding to TCF/LEF sites and inducing downstream gene transcription. TopFlash plasmid, used for β-catenin activation assays, encodes firefly luciferase under a promoter containing TCF/LEF binding sites. TopFlash and mCherry plasmid as a transfection controls. LiCl mimics the destruction complex inhibition, and was used as a positive control.

E. Activity of the transgenic NDP in TopFlash assay in HEK293 cells, co-transfected with NDP, and int. its receptor complex members and TopFlash plasmid. TopFlash plasmid, used for β-catenin activation assays, encodes firefly luciferase under a promoter containing TCF/LEF binding sites. TopFlash and mCherry plasmid as a transfection controls. LiCl mimics the destruction complex inhibition, and was used as a positive control.

F. Experimental design of the treatment administration, readouts and approximately corresponding stages in human development.

Eye and ear histology in all groups was analyzed at 2 months of age; a separate set of mice was analyzed for visual function at 1.5 months and audiology at 3 months of age.

FIG. 2 Expression of the construct in eye and ear at 2 months.

A. Weights of male mice before and after AAV administration and matched-age controls. Data are shown as mean±SD. Animal numbers: P2-L group and littermate controls, n (WT)=5, n(Ndp-KO)=2, n(P2-L)=16.

P30-H group and littermate controls, n(WT)=15, n(Ndp-KO)=5, n(P30-H)=14.

B-C. Transduction levels in P2 and P21 treated retinas: (B) P2-L group, n=4, (C) P30-H group, n=4. Staining: anti-GFP antibody and DAPI. GCL—ganglion cell layer, ONL—outer nuclear layer, INL—inner nuclear layer.

D. A schematic of the axial cross-section of one turn of the cochlea.

SL—spiral ligament, SV—stria vascularis, OoC—organ of Corti. Orange rectangle outlines region, shown in F.

E. Transduction in P2-treated mouse cochlea cross-section. SGN—spiral ganglion neurons.

F-G. Transduction of the lateral wall of the cochlea after treatment at P2 (G) and P30 (H). Scale bar: 500 μm, n=4.

H-I. Transduction of the organ of Corti transduction after treatment at P2 (H) and P30 (I). Scale bar: 100 μm.

FIG. 3 Effects of early and late treatment on the retinal vessel morphology and barrier, and the visual function.

Rescue of the retinal vascularization and visual function are limited to early treatment, meanwhile barrier factor expression remains responsive over time.

A, B. Schematic of the retinal vasculature in (A) cross-sections and (B) whole mounts. A′ indicates the depth projection colour scheme for C-F.

C-F. The three vascular plexuses in the central retina whole mounts, pseudocoloured depth projections in the central retina. Vasculature stained with isolectin B4 (IB4). Colour-depth scheme is depicted in A′. Treatment groups: C. WT, D. Ndp-KO, E. P2-L, F. P30-H. Arrows point to deep plexus vessels. Numbers 1, 2, 3 label the vascular plexuses. GCL—ganglion cell layer, INL—inner nuclear layer, ONL—outer nuclear layer.

G-J. Vasculature: IB4, nuclei: DAPI.

K-N. Colocalization of tight junction marker claudin-5 (CLDN5) and PLVAP.

O-S. Evaluation of visual function recovery with scotopic electroretinography (ERG) at 1.5 months of age.

O. Example of ERG waves at 10000 mcd/s·m\hat{ }2 flash intensity stimulus in WT, Ndp-KO, P2-L and P21-H groups, n=1.

P. Oscilatory potentials.

Q-R. ERG B-wave at increasing stimulus flash intensities in P2-L (Q) and P21-H (R) groups.

Data information:

Data are shown as individual trace in 0, and as mean±SD in P-R.

Sample numbers for C-N: n=4 per each group,

P analysed analysed with one-way ANOVA with Sidak's post hoc test, Q-R analysed with two-way repeated measures ANOVA with Tukey's post hoc test; all values compared to WT (blue asterisks) and Ndp-KO (red asterisks).

Post hoc test values: *P≤0.05, **P≤0.01, ***P≤0.001, ****P≤0.0001, ns—non-significant. Sample numbers for ERG analysis: n(WT)=10, n(Ndp-KO)=10, n(P2-L)=7, n(P21-H)=10.

FIG. 4: Restoration of the pathology-related gene expression in the cochlea by 2 months age demonstrated by RNA-seq and RT-PCR.

A Heatmap of the 45 pathology-related DEGs expression in WT, Ndp-KO and P-L group cochleas. This set of genes was not found to be significantly different in the WT versus P2-L comparison, meaning both that 1) no overinduction of the gene expression by treatment was identified and that 2) treatment restored expression of all genes to levels, comparable to the WT (side bar, dark blue). Within the 45 DEGs, 16 of the genes were also significantly different from the Ndp-KO, meaning their expression was restored to normal levels (purple), and 29 genes were significantly different only from the WT (light blue).

B-E qRT-PCR analysis of the expression of the pathology-related genes in all treatment groups. (B) Quality control of transgene (EGFP primers) expression. (C) Quality controls of mouse genotype by mouse Ndp expression. Expression of pathology related genes (D) Abcb1a, (E) Cldn5.

{Note that despite at least 10 times higher transgene levels in the P2-L group, the pathology-related genes are not upregulated to significantly higher levels than the WT}

Data information: B-E data are shown as mean SD;

Sample numbers: (A): n(WT)=4, n(Ndp-KO)=3, n(P2-L)=4,

(B-E): n(WT)=9, n(Ndp-KO)=12, n(P2-L)=10, n(P21-L)=8, n(P21-H)=6, n(P30-H)=8.

Statistical analysis: D-E analysed with one-way ANOVA with Sidak's post hoc test, all values compared to WT (blue asterisks) and Ndp-KO (red asterisks). Post hoc test values: *P≤0.05, **P≤0.01, ***P≤0.001, ****P≤0.0001, ns—non-significant.

FIG. 5: Differences of early (P2) and late (P30) treatment efficiency for the rescue of the cochlear vasculature by 2 months age.

A Schematic of the lateral wall vasculature. SL—spiral ligament, SV—stria vascularis capillaries.

B Quantification of capillary branching point numbers per area in 8 sequential regions along the stria vascularis.

Note, that branching was improved the in P2-L, but not in the P30-H groups. No significant morphological differences between WT and Ndp-KO nor other groups were detected in the rest of the cochlea.

C-F Capillary network density and morphology at the apical tip of the stria vascularis, anti-endomucin staining. Scale bar: 100 μm, n=4 per each group.

(C) WT, n=4, (D) Ndp-KO, n=4, (E), P2-L n=5, (F) P30-H, n=4.

Arrowheads: reduced network density and enlargement of vessel diameter.

G-J Capillary network density and tight junction marker claudin-5 expression in the spiral ligament (G-J) Anti-endomucin and (G′-J) anti-claudin-5 immunostaining. Scale bar: 100 μm, n=4 per each group. (G, G′) WT, (H, H′) Ndp-KO, (I, I′) P2-L, (J, J′) P30-H. Arrowheads: evenly branching vasculature with even expression of claudin-5 and endomucin in WT and P2-L samples. Arrows: types of diversified vessels. Red arrows: “meshwork” vessels. Orange arrows: “barrier” vessels.

Data information: B data are shown as mean±SD.

Statistical analysis: two-way repeated measures ANOVA with Tukey's post hoc test, all values compared to WT (blue) and Ndp-KO (red).

Post hoc test values: *P≤0.05. **P≤0.01, ns—non-significant.

FIG. 6: Rescue or improvement of the outer hair cell survival in all treatment groups by 2 months.

A-E: Examples of hair cell survival in matching “sensitive” regions along the tonotopic axis from different treatment groups. (A) WT, n=7, (B) Ndp-KO, n=6, (C) P2-L, n=5, (D) P21-H, n=6, (E) P30-H, n=8.

F-K: Quantification of the surviving hair cells from the same samples as in A-E and P21-L group. Analysed with repeated measures two-way ANOVA with Tukey's post hoc test, samples compared to the WT. (F) WT, n=3, (G) Ndp-KO, n=6, (H) P2-L, n=5, (I) P21-L, n=3, (J) P21-H, n=6, (K) P30-H, n=8.

Data information: Quantification data are shown as mean±SD.

Analysed with two-way repeated measures ANOVA with Tukey's post hoc test. Significant effects of region (P≤0.0001), treatment group (P≤0.0001) and their interaction (P≤0.0001).

Post hoc test values: *P≤0.05, **P≤0.01, ***P≤0.001, ****P≤0.0001, ns—non-significant.

FIG. 7: Rescue or improvement of audiological readouts and hearing at 3 months.

(A) Endocochlear potentials (mean±SD), analysed with one-way ANOVA with Sidak's post hoc test, each group compared to WT. P (Ndp-KO vs WT)=0.0107.

(B) Overlay of DPOAE threshold averages. Grey area marks region in the WT, affected by C57BL/6-related degeneration.

(C) Click ABR thresholds (mean±SD), analysed with one-way ANOVA with Sidak's post hoc test, each group compared to WT.

(D) Overlay of Pure tone ABR threshold averages from all groups. Grey area marks region in the WT, affected by C57BL/6-related degeneration.

(E) Schematic of tonotopic region correspondence with audiology measures. The whole length of the cochlea is divided in 8 equal length regions (⅛-8/8) Black arrowheads mark the respective frequencies (kHz), to which specific points corresponds. Red line indicated the tonotopic region, sensitive to degeneration in Ndp-KO on C57BL/6 strain. Grey lines indicate frequency regions, in which the ABR and DPOAE were recorded. Grey area indicates the C57BL/6 strain-related region, which degenerates in the WT.

(F) Schematic of the putative Norrie phenotype rescue mechanism by gene therapy. Green colour labels the typically transduced green in all treatment groups, red arrows indicate the putative targeting of the NDP, produced in and secreted from the transduced areas.

Data information: Data are shown as mean±SD in A and C, and as mean in B, D. N numbers are as indicated in each key.

Animal numbers for audiology analyses:

Endocochlear potential:

n(WT)=8, n(Ndp-KO)=6, n(P2-L)=6, n(P21-H)=6, n(P30-H)=7.

DPOAE:

n(WT)=11, n(Ndp-KO)=6, n(P2-L)=9, n(P30-H)=8.

Click and pure tone ABR:

n(WT)=12, n(Ndp-KO)=7, n(P2-L)=10, n(P21-H)=8, n(P30-H)=8.

Statistical analysis for B and D is provided in the supplementary image.

Post hoc test values: *P≤0.05, **P≤0.01, ***P≤0.001, ****P≤0.0001, ns—non-significant.

FIG. 8: Detection of the transgenic proteins by Western blot in the lysates of HEK293 cells, transfected with the construct.

Samples processed under different conditions. HI: Heat inactivation, R: reduction, Tr: transfection.

(A) EGFP is detected as an ˜29 kDa band in non-reducing conditions and 25 kDa in reducing conditions (arrowheads).

(B) Loading control (GAPDH).

(C) Anti-FLAG antibody two NDP bands on the same blot (arrowheads): 15 kDa-size monomer and >250 kDa oligomer or aggregate.

(D) Anti-NDP antibody also detects NDP monomers or oligomers. Note that anti-flag or anti-NDP staining does not colocalize with GFP, and the molecular weights of the monomers are of expected size, meaning that no fusion protein was produced.

(E) Loading control (GAPDH)

FIG. 9: A-B. Weights of mice before and after AAV administration at P21 and matched-age controls. Data are shown as mean±SD.

(A) Ctrl N=7, P21-L dose N=14.

(B) Ctrl N=14 P21-H dose, N=9.

FIG. 10: Degree of vascular coverage at the time of treatment (P2) and resulting AAV9 vector transduction in the retina.

(A,B) Flatmounts of WT and Ndp-KO retinas at P2, vasculature immunostained with anti-endomucin

(C,D) Cryosections of WT and Ndp-KO retinas at P21, vasculature immunostained with isolectin-B4. Scale bar=50 μm.

(E,F) Flatmounts of WT and Ndp-KO retinas at P30, vasculature immunostained with anti-endomucin. Scale bar=500 μm.

(G,H) GFP staining at 1 month in retina after treatment at P2 (G) and P21 (H).

FIG. 11: AAV9 vector transduction in the cochlea. Organ of Corti and lateral wall wholemounts stained with anti-GFP antibody.

(A-A″) Untreated cochlea

(B-B″) P2-L dose

(C-C″) P21-L dose

(D-D″) P21-H dose

(E-E″) P30-H dose

Scale bars=500 μm (A,B,C,D,E) 500 μm (A′,B′,C′,D′,E′)

FIG. 12: Electroretinograms showing effects of early and late treatment

(A) Representative ERG traces in response to single flashes of light of increasing intensity.

(B,C) Flash ERG, ratio of b wave to a wave amplitude

(D,E) Flash ERG, a wave amplitudes

WT N=10, Ndp-KO, N=10, P2-L N=7, P21-H N=10

FIG. 13: Differential gene expression in the cochlea by RNA sequencing and qPRC

(A) Venn diagramme showing genes differentially expressed between Ndp-KO and WT cochleas and between Ndp-KO and P2-L cochleas

(B) Genes found to be significantly differentially expressed between the Ndp-KO and P2-L cochleas. Note that these genes showed a similar pattern of expression between Ndp-KO and WT cochleas but the fold changes did not reach significance. No gene were found to be significantly differentially expressed between WT and P2-L cochleas.

(C,D) Expression patterns of Plvap and Sox17 in all groups by qPCR. WT N=9, Ndp-KO N=12, P2-L N=10, P21-L N=8, P21-H N=6, P30-H N=8

FIG. 14: Hair cell survival along the apex to base axis in organ of Corti wholemounts. Regions of the cochlea and corresponding frequencies are shown at the top of the figure.

(A) WT N=3

(B) Ndp-KO N=6

(C) P2-L N=5

(D) P21-LN=3

(E) P21-HN=6

(F) P30-H N=8

Scale bar=500

FIG. 15: Statistical analysis of DPOAE and ABR thresholds for all groups.

(A, B) DPOAE thresholds of P2-L, P-30H and control groups. P values indicate statistically significant difference from WT.

(C-E) ABR thresholds of P2-L, P-30H and control groups. P values indicate statistically significant difference from WT.

Data information: Data are shown as mean±SD. N numbers are as indicated in each key.

Statistical analysis was performed for each group individually, comparing treated samples with the same WT and Ndp-KO control groups. Post hoc test values: *p≤0.05, **p≤0.01, ***p≤0.001, ****p≤0.0001.

FIG. 16: Experimental design of experiment for systemic expression of codon-optimised NDP after intravenous injection.

FIG. 17: Experimental design of experiment for intraocular expression of codon-optimised NDP after intraocular injection.

FIG. 18: Intravitreal injection of AAV2/ShH10/NDP at p22 rescues the blood retinal barrier (using experimental (c0) and clinical (c6) (codon-optimised)) constructs).

A: BSA-AF 549 concentration is linearly related to fluorescence in the dynamic range relevant to this experiment.

B: Box and whisker plot showing the degree of blood retinal barrier (BRB) transcellular permeability in the WT, Ndp, KO and AAV2/ShH10.NDP KO. Each circle represents a single data point.

FIG. 19: Cross-section of the retina from an Ndp KO mouse at p30, which had been treated with intravitreal construct 6 (SEQ ID NO 21, SEQ ID NO: 22) (3.47×109 vector genomes per eye) at p22.

FIG. 20: Cross-sections of the retina of a WT and Ndp KO mice at p30.

FIG. 21: Sections showing retinol vasculature—1 layer only in Ndp-KO compared to 3 in WT.

FIG. 22: Intravitreal injection of AAV2/ShH10.NDP at p8 rescues the intermediate and deep retinal capillary layers.

FIG. 23: Methods of vessel analysis—retinal flat mounts.

FIG. 24: Peripheral vascular density in p8 or p21 BSS-treated WT mice, BSS-treated Ndp KO mice and AAV2/ShH10-treated Ndp KO mice as assessed at p30.

FIG. 25: Quantification of radius of peripheral vascularisation.

FIG. 26: The radius of peripheral retinal vascularisation was increased by intravitreal treatment with construct 0 (SEQ ID NO 18, SEQ ID NO: 19) at P8 but not at P21.

FIG. 27: The b wave height can be partially rescued with intravenous delivery of AAV2/9.NDP at p2 but not p21.

FIG. 28: The b wave height can be rescued with intravenous delivery of AAV2/9.NDP (construct 0 (SEQ ID NO 18, SEQ ID NO: 19)) at p2 but not p21.

FIG. 29: Schematic diagram of Norrie disease progression.

FIG. 30: Schematic showing cochlear (A) and retinal (B, C) vasculature.

FIG. 31: Retina images of a WT and Ndp KO mice (A, B) at P2 and retina images (C, D) and cross-sections of the retina (E, F) at 1 month.

FIG. 32: Development of vascular pathology in WT and Ndp KO mice.

FIG. 33: Schematic diagram of therapeutic window.

FIG. 34: Schematic representation of NDP transduced and receptive cells

FIG. 35: Schematic diagram of construct packaging into an AAV vector, using a triple transfection protocol.

FIG. 36: Construct labelled the transduced HEK293 cells. Anti-flag tag immunostaining colocalized with the intrinsic GFP.

FIG. 37: Western blot analysis detected expression of NDP protein monomer and GFP as separate proteins.

FIG. 38: A) Schematic representation showing that Norrin is a secreted signalling protein, which induces canonical WNT/0-catenin signaling through FZ4 complex with LRP5 or LRP6 and TSPAN12. B) graph showing the ability of the construct to induce β-catenin signalling through these receptors was demonstrated in vitro using a TopFlash assay.

FIG. 39: Graph showing that Ndp-KO mice, treated at neonatal age, grew and developed as normal.

FIG. 40: Retinal vascular morphology of treated Ndp-KO mice was comparable to the WT.

FIG. 41: Cochlear vascular morphology of treated Ndp-KO (C) mice was comparable to the WT.

FIG. 42: HEK293 cells transduced with NDP construct (CE10).

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated.

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients. Suitable assays to measure the properties of the molecules disclosed herein are also described in the examples.

The present invention is a construct comprising a wildtype NDP nucleic acid sequence or a codon-optimised NDP nucleic acid sequence.

Human patients with a mutation in the gene NDP may develop retinal and cochlear disease, causing loss of vision and hearing.

Norrie disease, caused by NDP gene mutation, is a severe X-linked disorder causing blindness and progressive deafness; about 30% of patients also have cognitive impairment and peripheral vascular disease. The blindness results from disruption of retinal vascular development causing hypoxia, ischaemia, persistent foetal vasculature and compensatory neovascularization. Hearing appears normal at birth, and boys with Norrie pass Newborn Hearing Screening. However, usually beginning in adolescence, nearly all boys develop progressive hearing loss. The present invention is an NDP genetic therapy construct, consisting of a functioning copy of the NDP gene within an adeno-associated viral vector (NDP.AAV), that has the potential to prevent loss of vision and/or hearing.

The use of AAV as a vector for gene therapy is in common use experimentally and is also the basis for several commercially available gene therapies (for example as for RPE65-related Lebers' Congenital Amaurosis).

Loss of vision and hearing originate from (i) insufficient retinal and cochlear vascularisation during foetal development, (ii) poor function of the blood vessel barrier. As demonstrated here, administration of NDP.AAV according to the present invention during vascular development in the Ndp knockout mouse can partially rescue peripheral retinal vascularisation in the retina and can reduce outer hair cell loss in the cochlea (hair cell loss causes deafness). In addition, administration even once vascular development is complete can partially restore the blood-retinal barrier in the NDP knockout mouse.

Delivery of NDP.AAV has been effective by both the intravenous and intravitreal (intraocular) routes. For intravenous injections, the adenovirus used was AAV 2/9, for the intraocular injections AAV 2/ShH10 was used. AAV 2/ShH10 was chosen for intraocular delivery as highly efficient transfection of muller cells (the natural site of NDP expression in the retina) has previously been demonstrated.

Expression of NDP results in the secretion of functional norrin protein, which is a signalling protein within the extracellular matrix of the retina and cochlea. NDP is necessary for the functioning of the WNT signalling pathway that results in normal retinal and cochlear vascularisation and proper functioning of the blood-retinal barrier in the eye/maintenance of the endocochlear potential in the cochlea.

In one embodiment the construct may express the wildtype NDP nucleic acid sequence. In another embodiment, the construct may express a codon-optimised NDP nucleic acid sequence. The wildtype and codon-optimised NDP nucleic acid sequences may be human NDP nucleic acid sequences. For example, in one embodiment, the wildtype NDP nucleic acid sequence may be the nucleotide sequence of SEQ ID NO: 1. The polypeptide sequence expressed from SEQ ID NO. 1 is shown in SEQ ID NO: 2. In an alternative embodiment, the codon-optimised NDP nucleic acid sequence may be the nucleotide sequence of SEQ ID NO: 3. The polypeptide sequence expressed from SEQ ID NO. 3 is shown in SEQ ID NO: 4.

In the present invention, the codon-optimised NDP sequence of SEQ ID NO: 3 is shown to be particularly effective at rescuing sight and hearing loss as part of an AAV gene therapy construct.

In a further alternative embodiment, the construct may comprise a nucleic acid sequence which has at least about 90% or 91% or 92% or 93% or 94% or 95% or 96% or 97% or 98% or 99% sequence identity to SEQ ID NO: 1.

In a yet further alternative embodiment, the construct may comprise a nucleic acid sequence which has at least about 90% or 91% or 92% or 93% or 94% or 95% or 96% or 97% or 98% or 99% sequence identity to SEQ ID NO: 3.

In some embodiments, the NDP nucleic acid sequence may be a DNA, RNA, cDNA, or PNA and may be recombinant or synthetic. It may be single stranded or double stranded.

In some embodiments the NDP polypeptide expressed from the NDP nucleic acid sequence is in the secreted form of the NDP polypeptide.

In one embodiment, the construct may comprise at least a promoter sequence, for example a CAG promoter sequence or a CBA promoter sequence, full length human NDP coding sequence (402 bp including the native secretion signal), and a WPRE sequence.

In one embodiment, the construct may comprise at least a promoter sequence, for example a CAG promoter sequence or a CBA promoter sequence, full length human NDP coding sequence (402 bp including the native secretion signal), a WPRE sequence, and SV40 late poly A sequence at the 3′end.

In other embodiments, the promoter is CAG promoter, a CBA promoter, CMV promoter, EF T a promoter, PGK promoter, TRE promoter, U6 promoter, UAS promoter, EFS promoter, SFFV promoter, MSCV promoter, SV40 promoter, UBC promoter, Pro1A promoter, hRHO promoter, hBEST1 promoter, Grm6 promoter, GJB2 promoter, a GJB6 promoter, a SLC26A4 promoter, a TECTA promoter, a DFNA5 promoter, a COCH promoter, a NDP promoter, a SYN1 promoter, a GFAP promoter, a PLP promoter, a TAK1 promoter, a SOX21 promoter, a SOX2 promoter, a FGFR3 promoter, a PROX1 promoter, a GLAST1 promoter, a LGR5 promoter, a HES1 promoter, a HES5 promoter, a NOTCH1 promoter, a JAG1 promoter, a CDKN1A promoter, a CDKN1B promoter, a SOX10 promoter, a P75 promoter, a CD44 promoter, a HEY2 promoter, a LFNG promoter, a S100b promoter, a CLDN11 promoter, an NDP promoter, or synthetic modifications or combinations of these promoters.

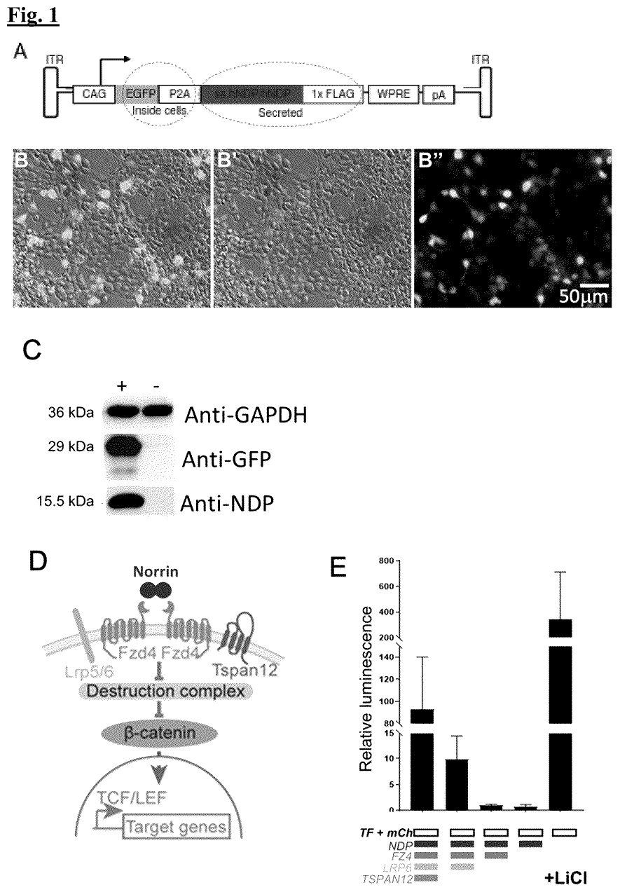

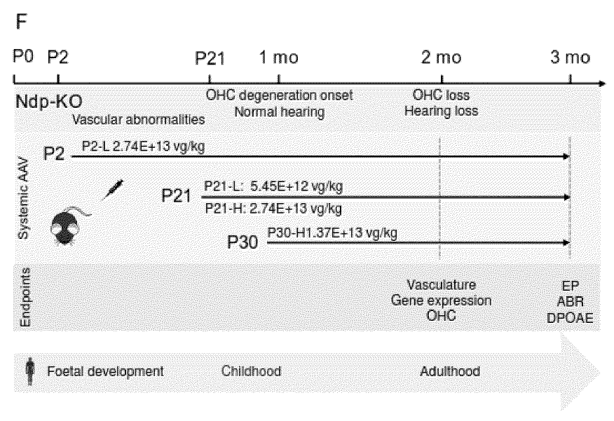

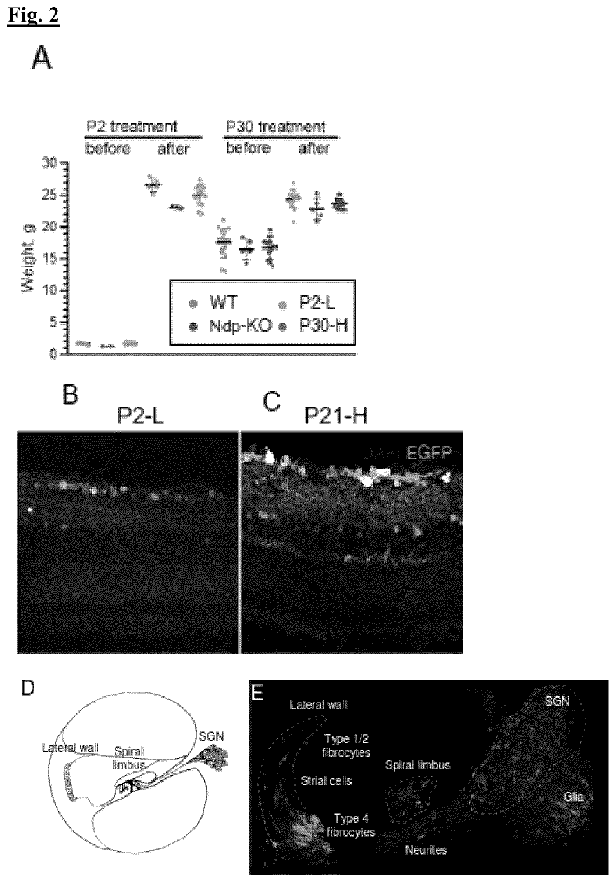

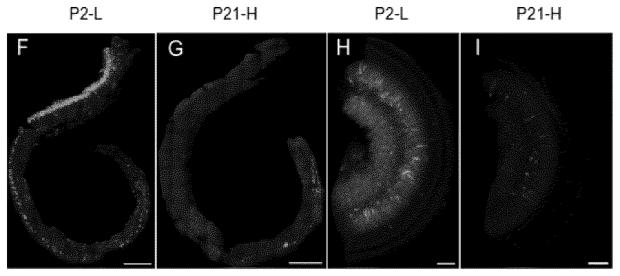

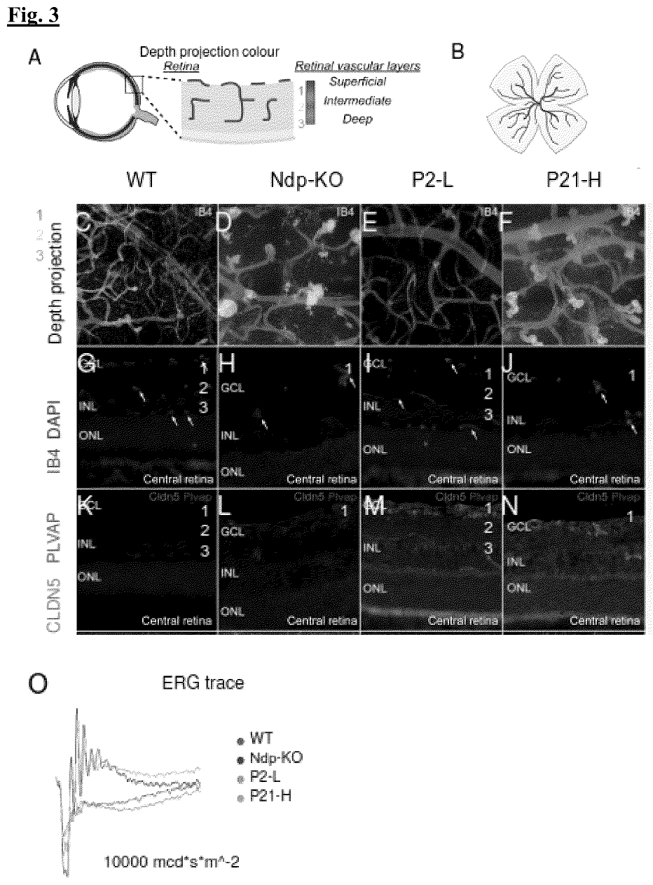

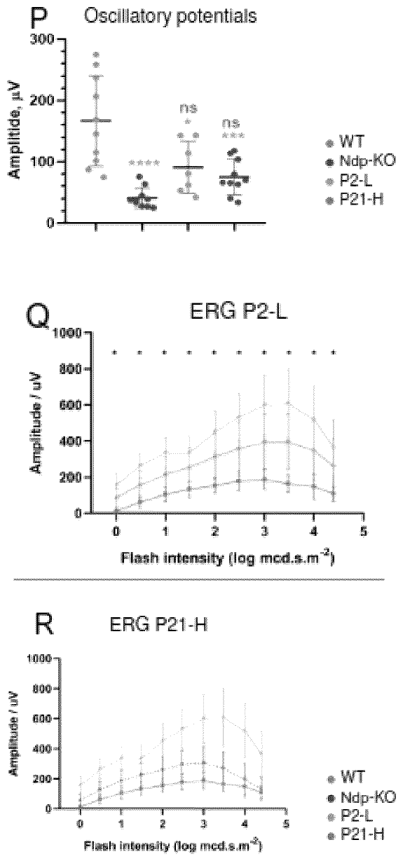

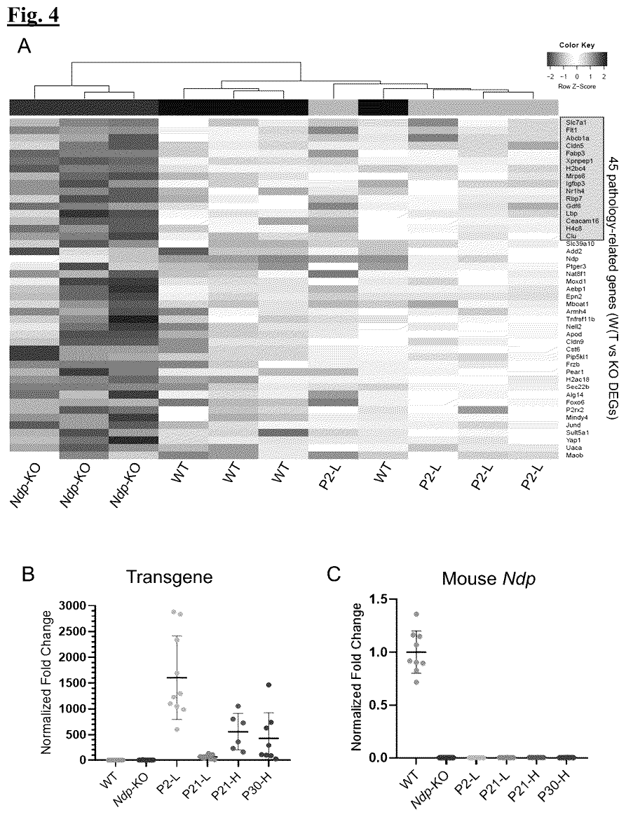

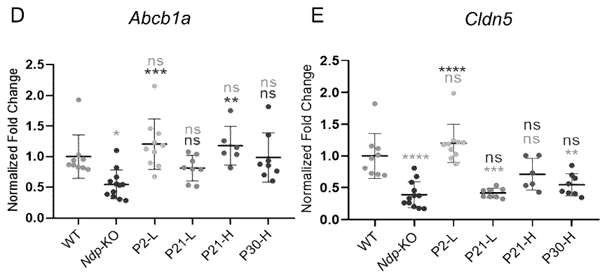

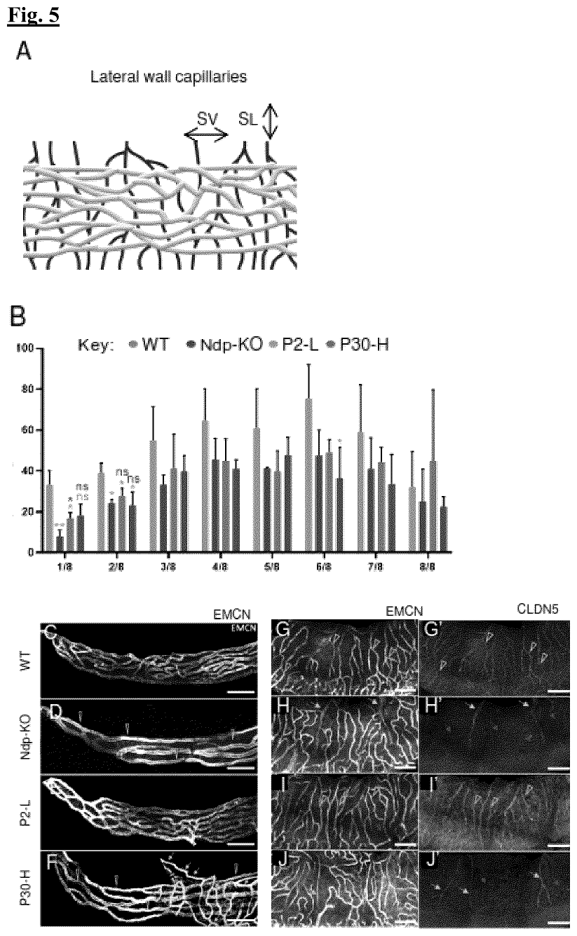

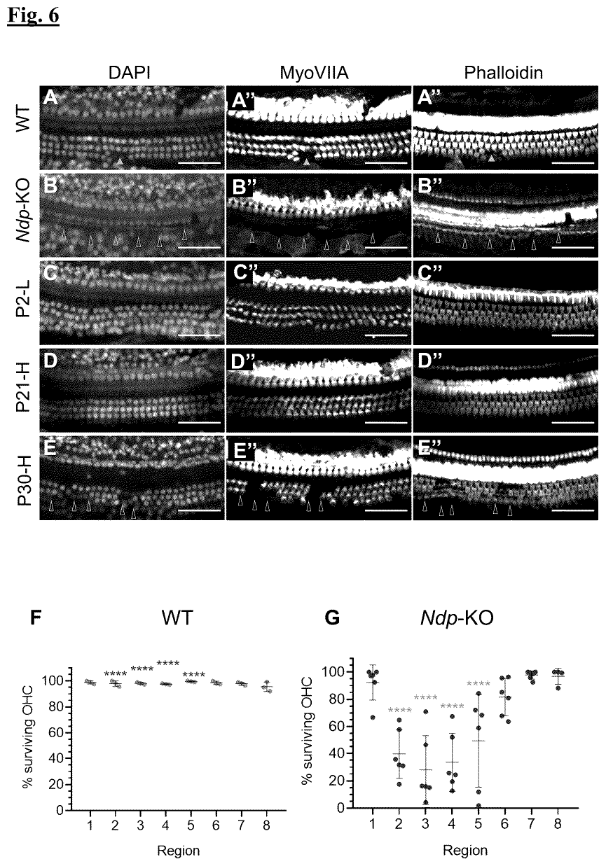

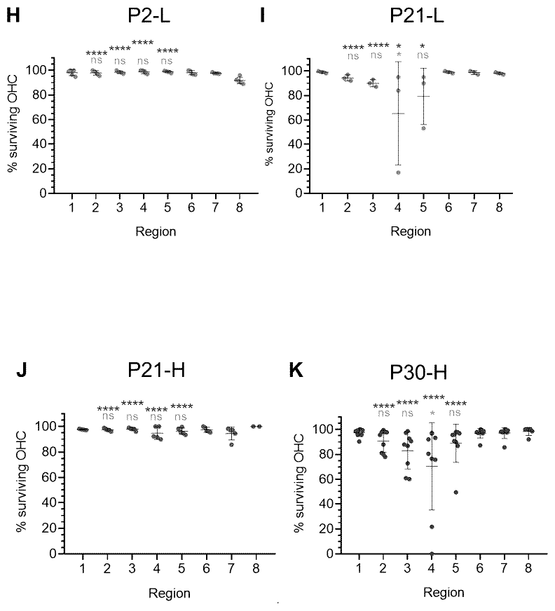

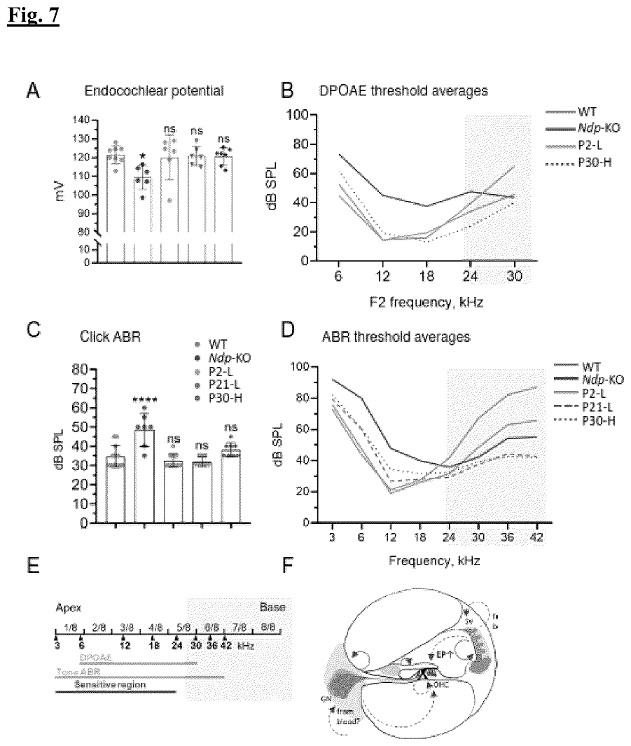

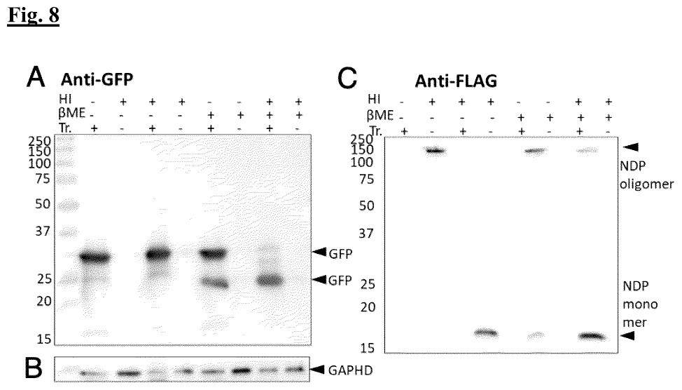

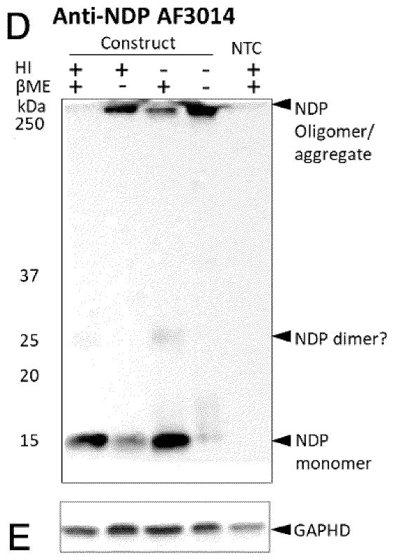

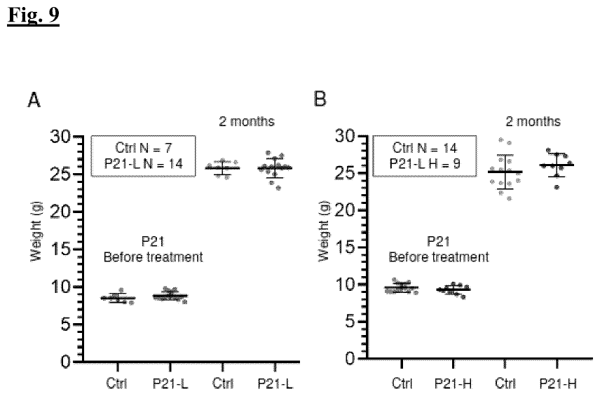

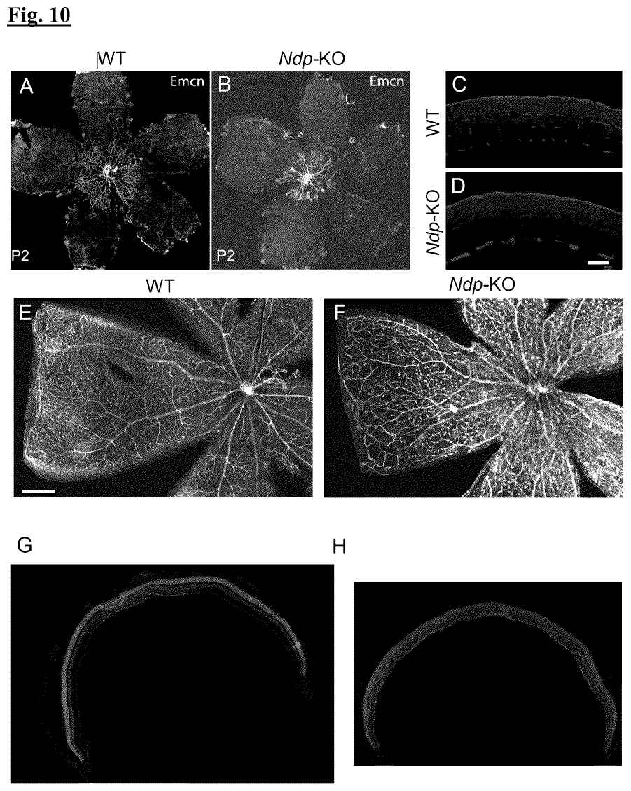

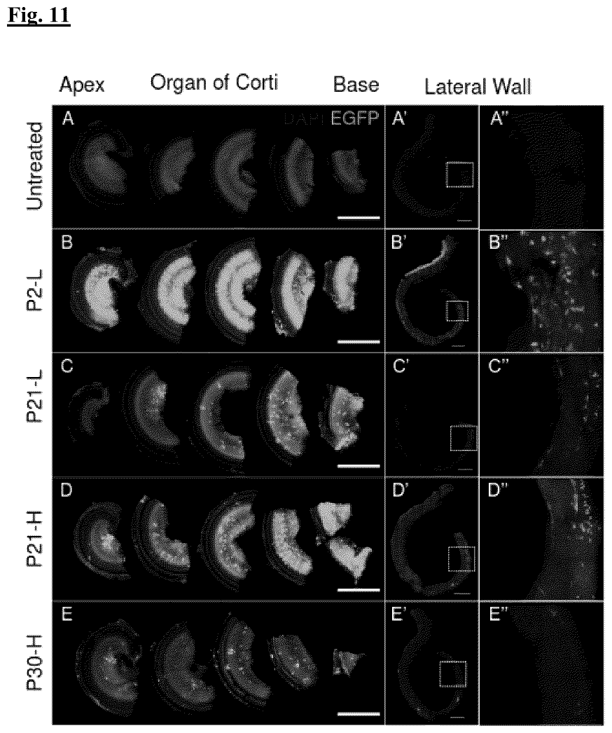

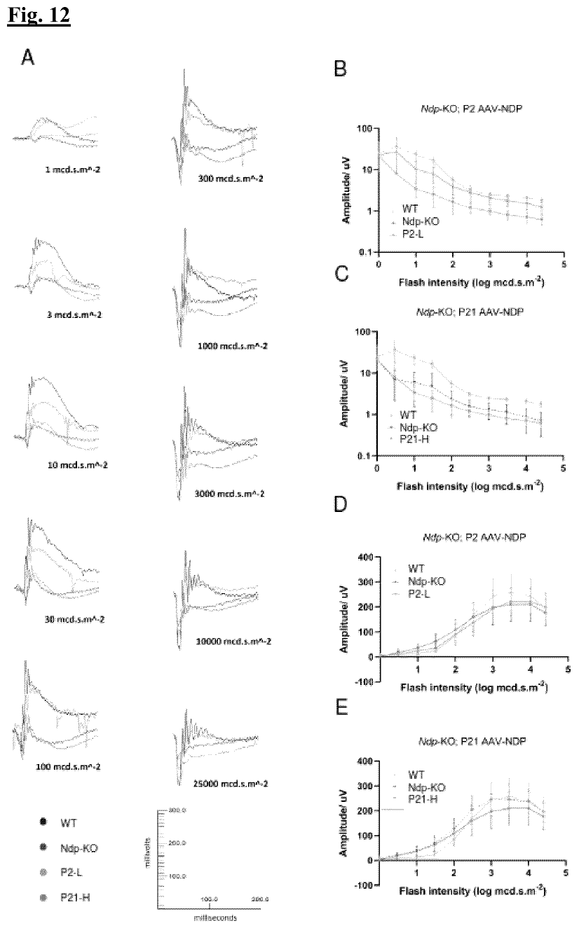

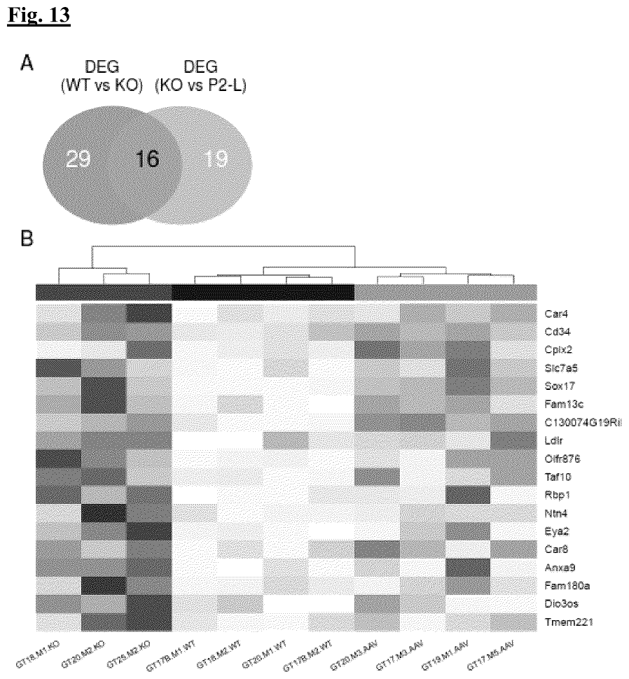

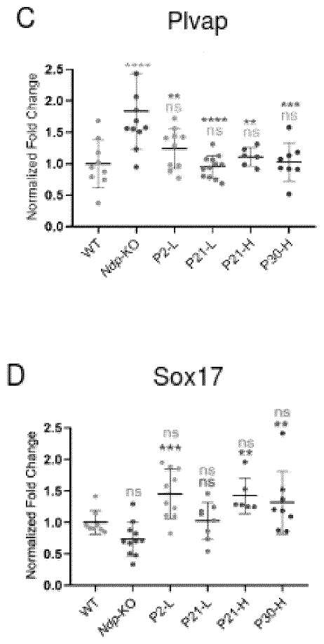

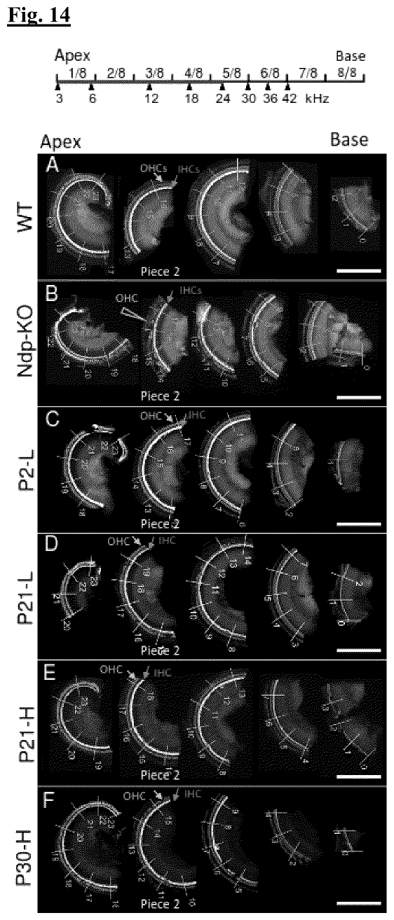

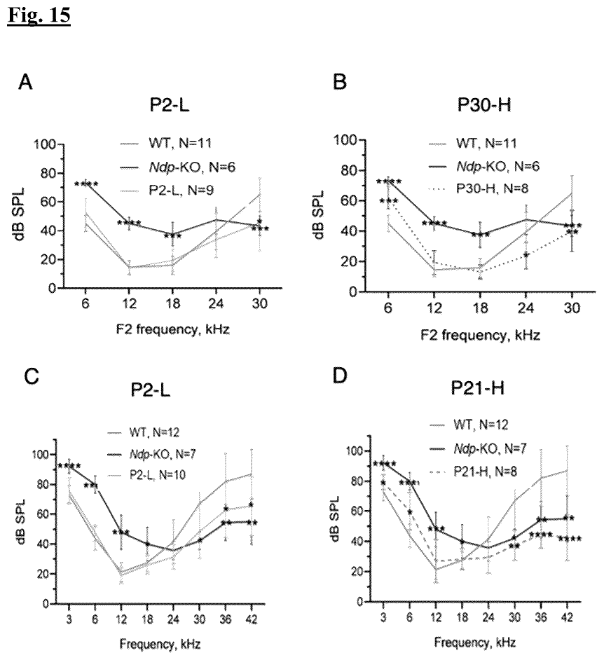

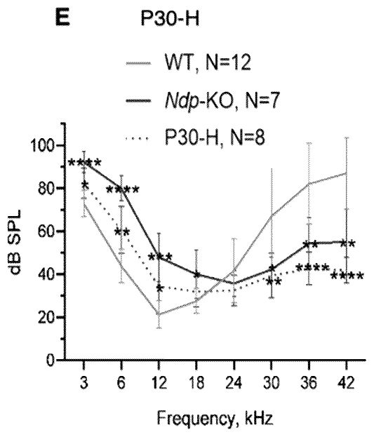

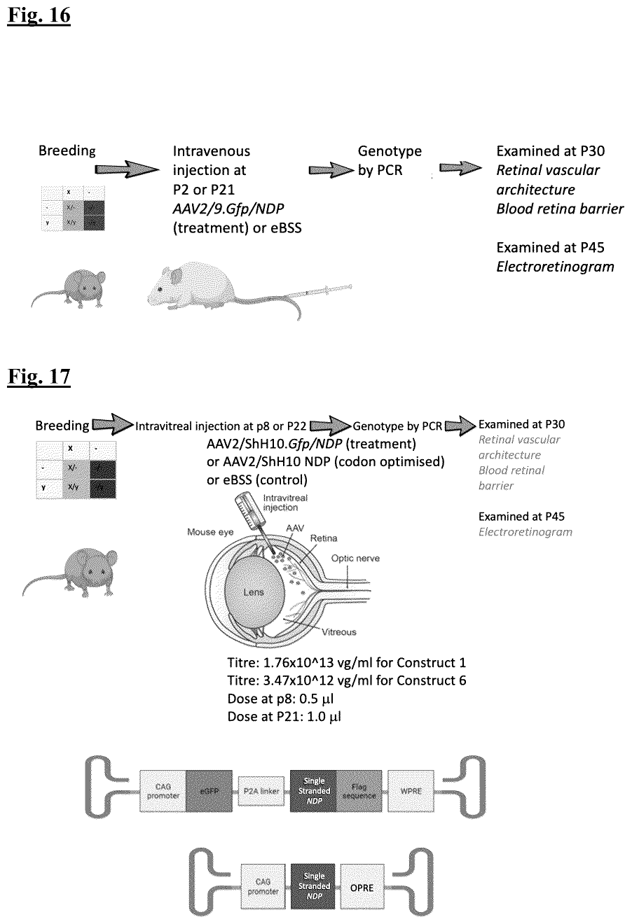

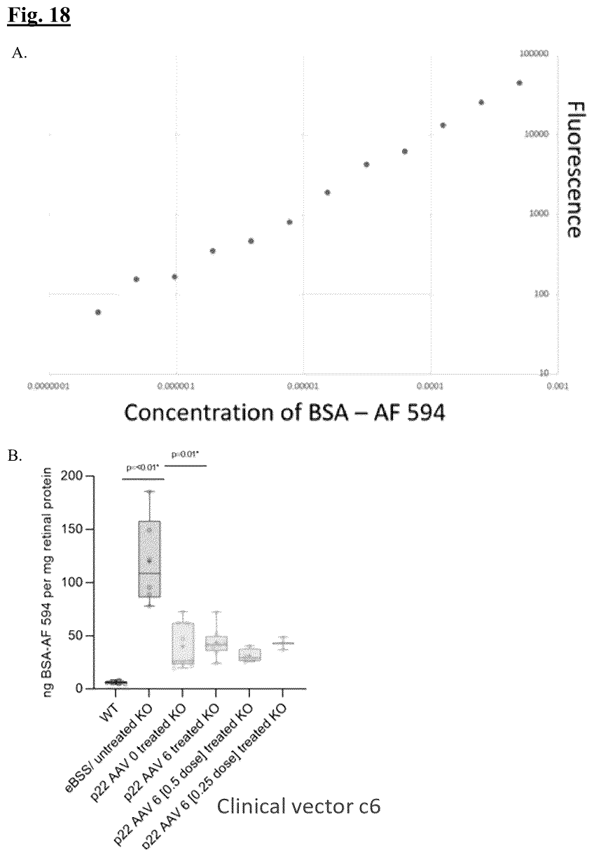

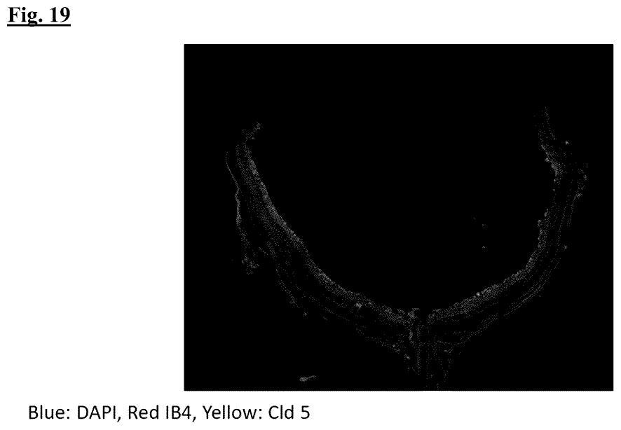

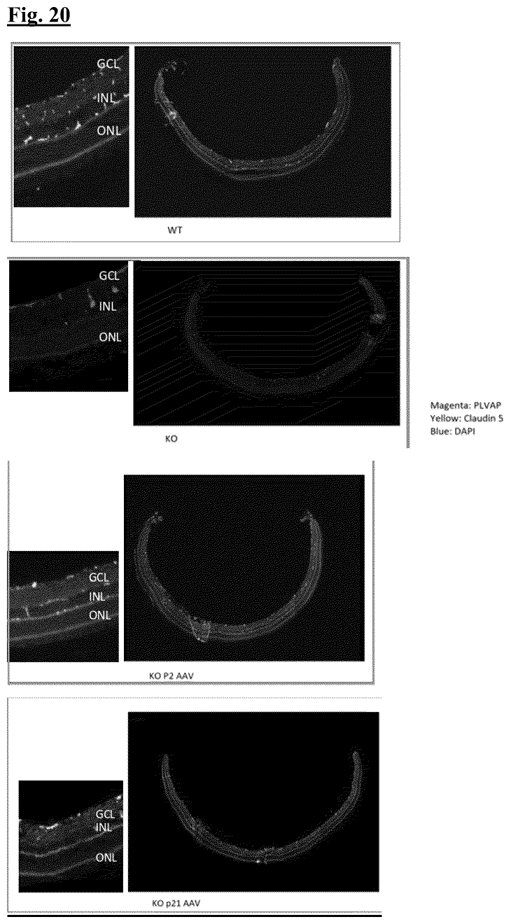

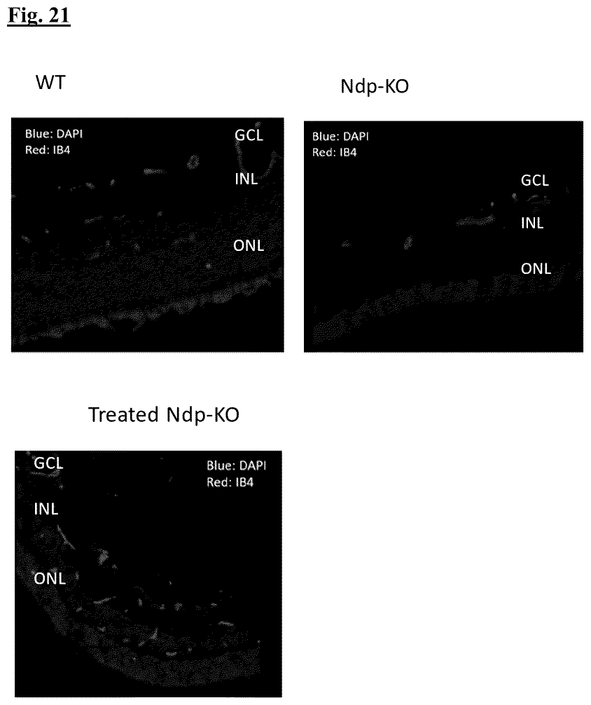

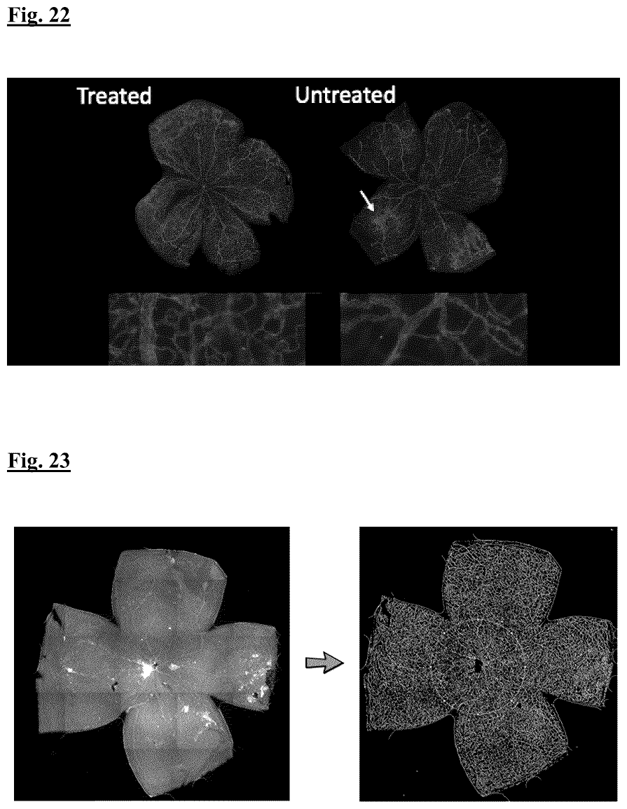

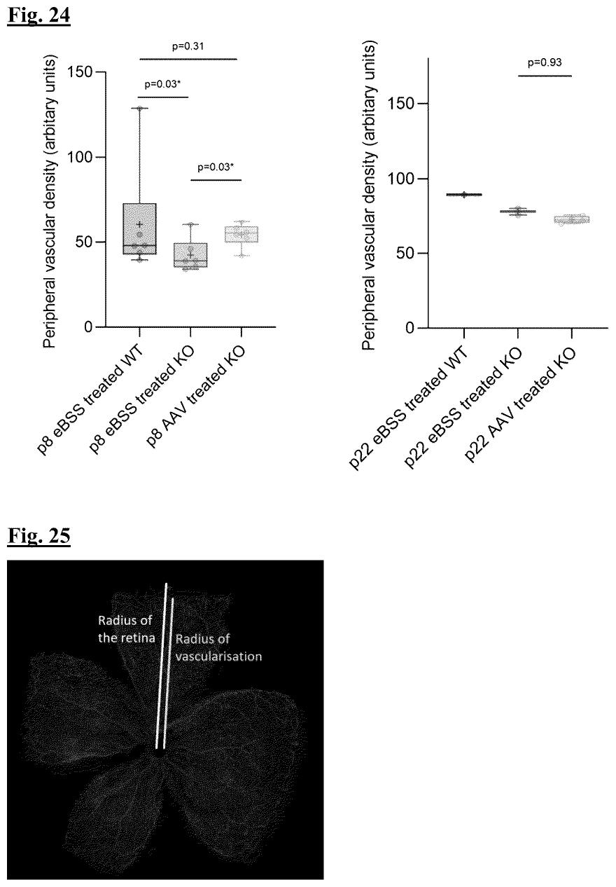

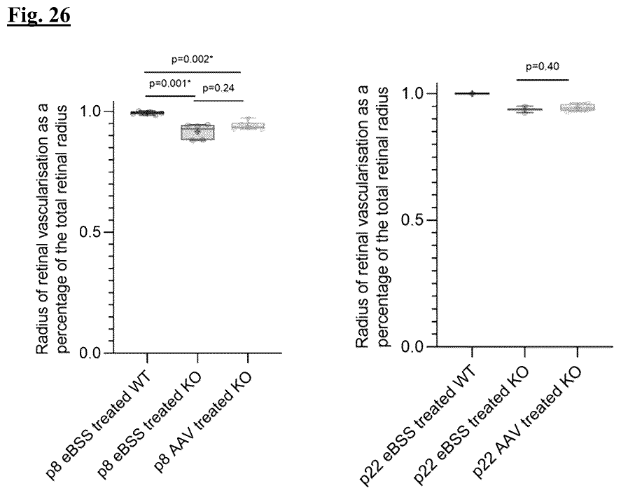

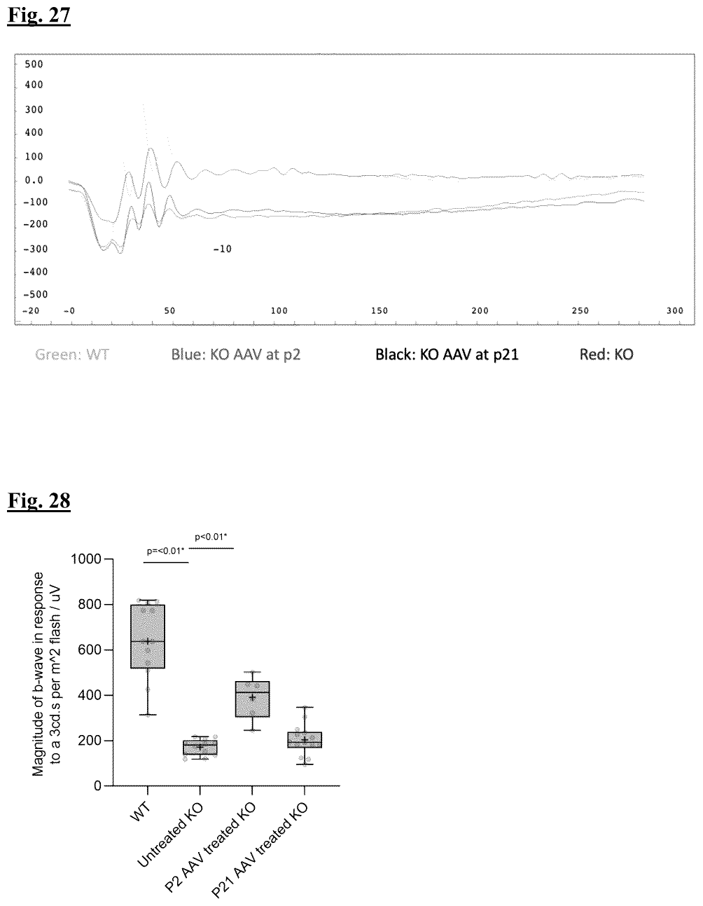

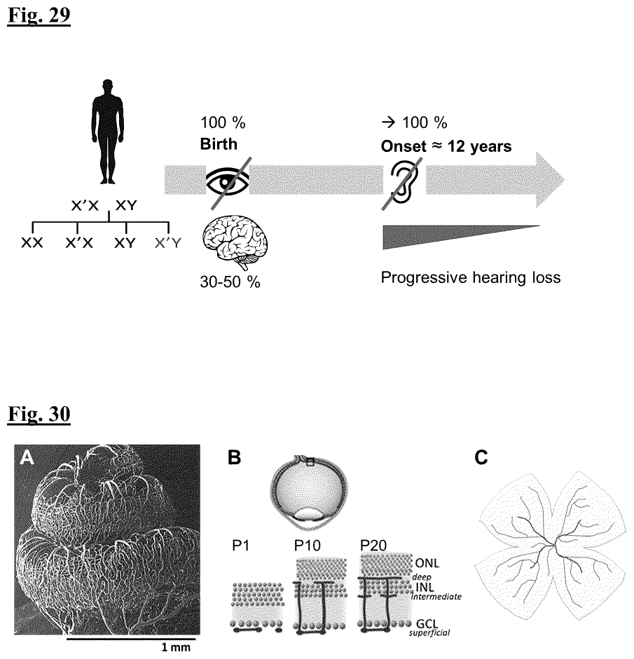

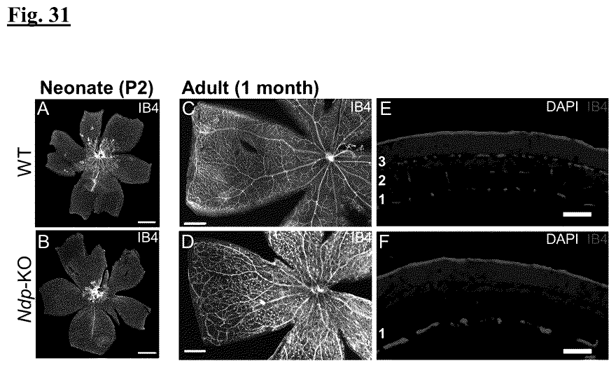

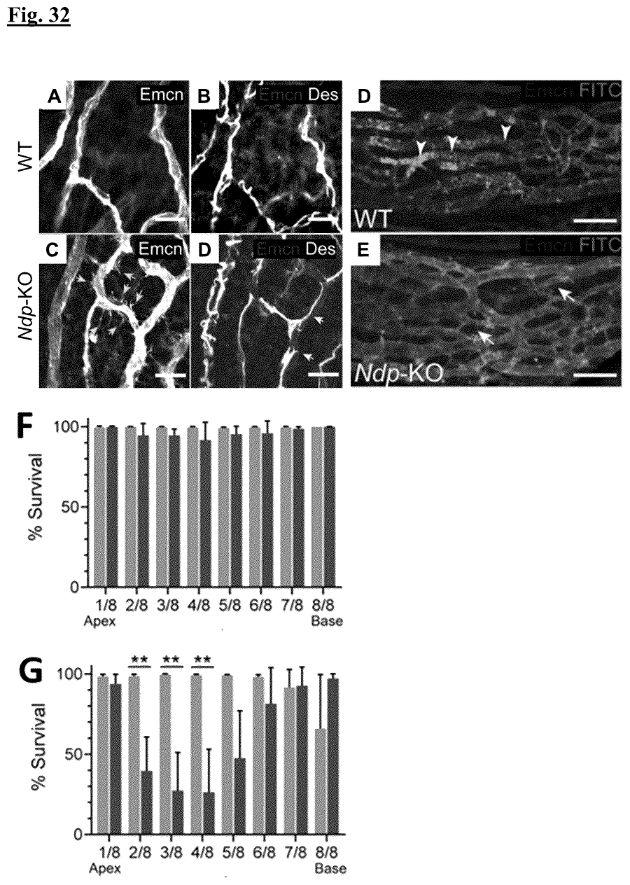

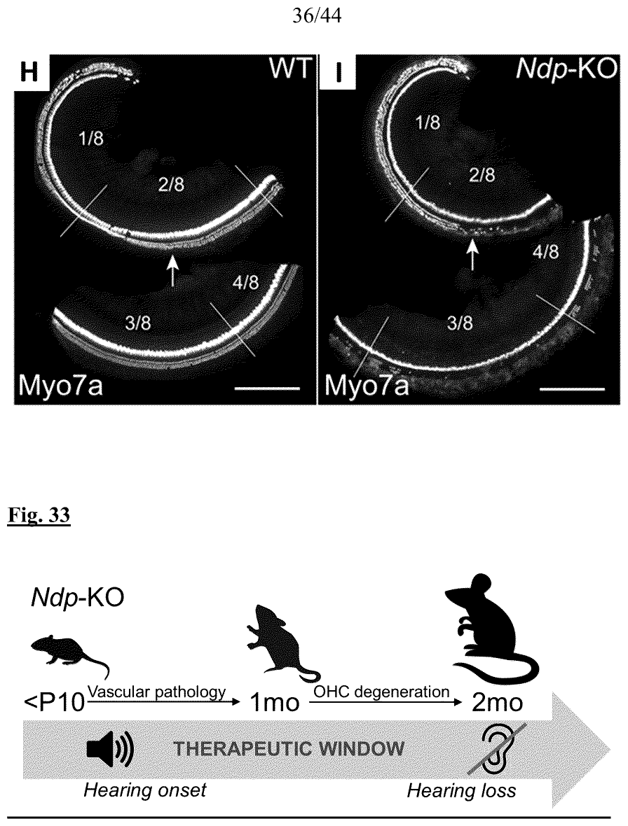

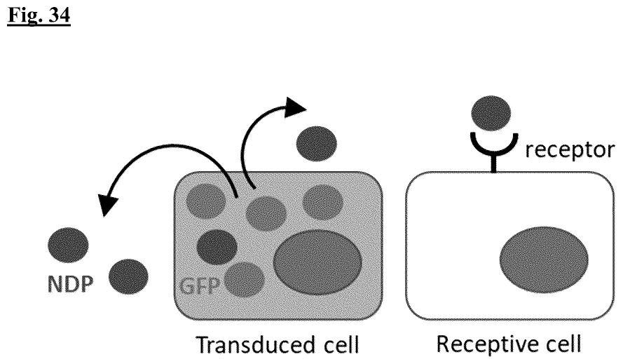

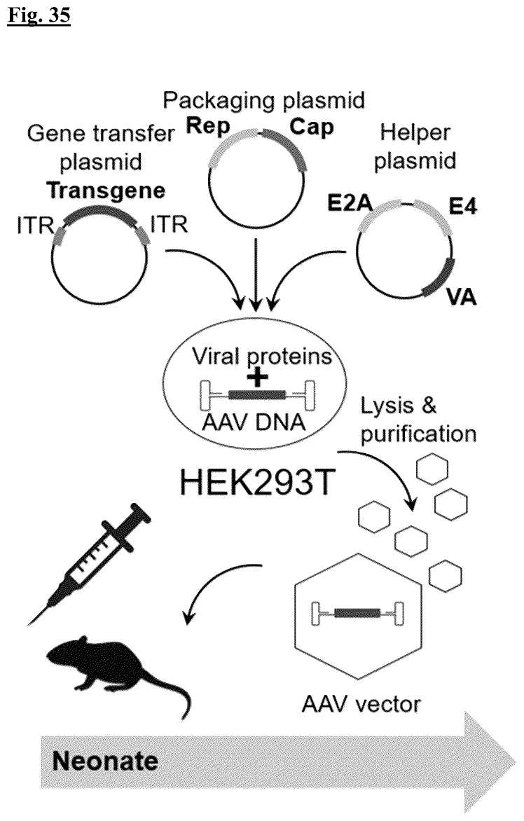

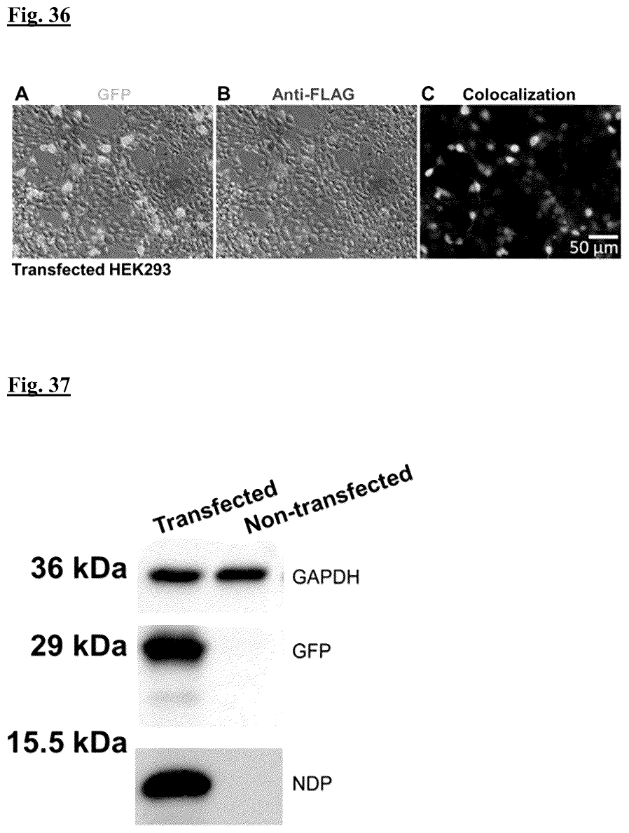

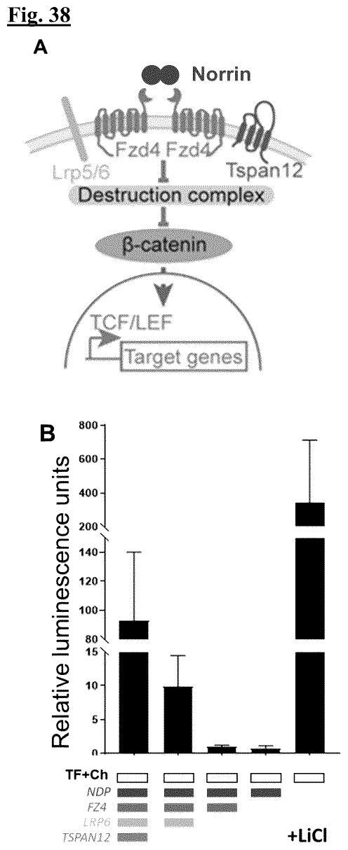

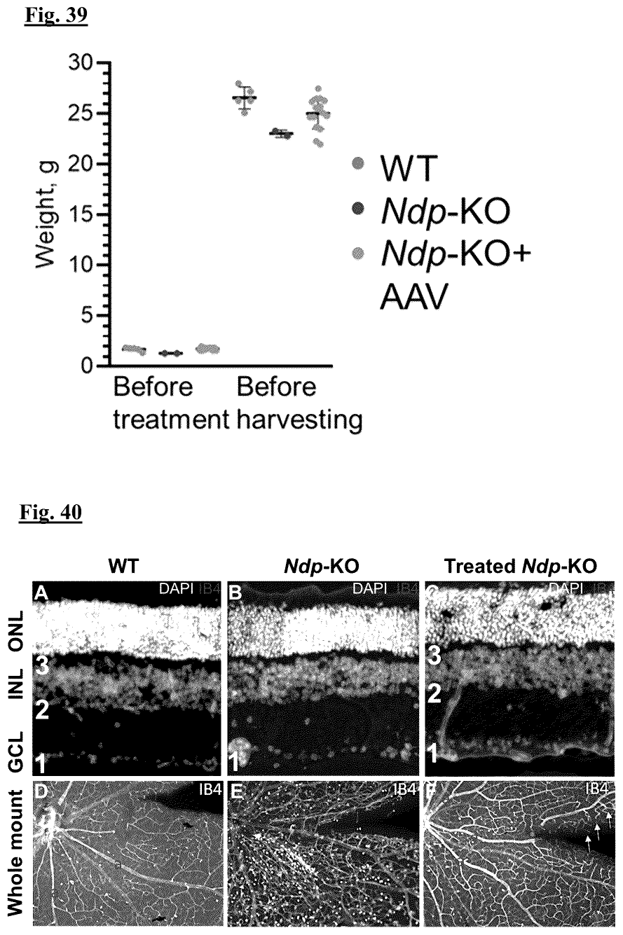

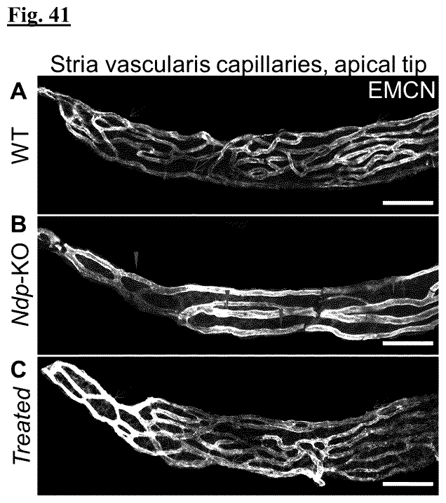

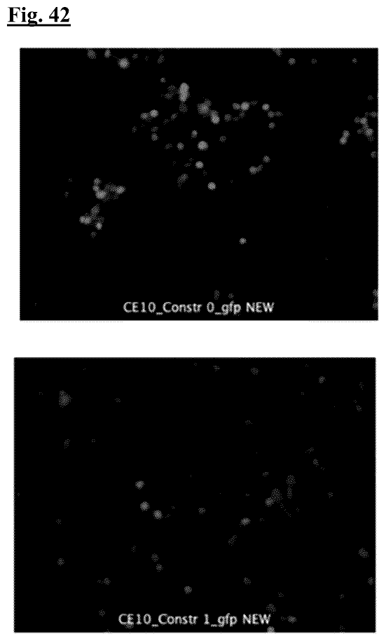

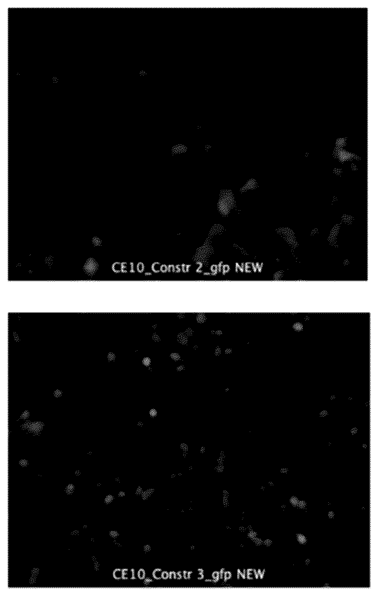

In the embodiment shown in FIG. 1A, the construct may comprise the following elements of the strong ubiquitous CAG promoter upstream of EGFP (to label the transduced cells), a self-cleaving P2A linker, full length human NDP coding sequence (402 bp including the native secretion signal) with a FLAG epitope sequence tagging the C terminus to aid detection of transgenic norrin, followed by a WPRE sequence and SV40 late poly A sequence at the 3′end. In some embodiments the construct may comprise an AAV vector. For example, the construct may comprise one or more of the following: AAV1, AAV2, AAV3, AAV4, AAV5, AAV6, AAV7, AAV8, AAV9, AAV10, AAV-rh8, AAV-rh10, AAV-rh39, AAV-rh43, AAVAnc80, AAV 2/ShH10, AAV-S vector.

In a preferred embodiment, the wildtype NDP nucleic acid sequence or the codon-optimised NDP nucleic acid sequence is incorporated into an AAV 2/ShH10 vector. The NDP expressed from a AAV 2/ShH10 and AAV-S vector is advantageous for administration intraocularly.

In another preferred embodiment, the wildtype NDP nucleic acid sequence or the codon-optimised NDP nucleic acid sequence is incorporated into an AAV 2/9 vector. The NDP expressed from a AAV 2/9 and AAV-S vector is advantageous for administration intracochlearly.

An AAV particle (NDP.AAV) comprising the construct may be used for use in the treatment of one or more of Norrie disease, age related hearing loss, diabetic maculopathy and retinopathy, retinal neovascularisation, retinal exudation, retinopathy of prematurity (ROP), Familial exudative vitreoretinopathy (FEVR), Coats disease, and other NDP-related diseases, or other beta catenin signalling-related retinal diseases.

An AAV virion carrying the NDP gene may be introduced by injection into an ear or eye of a patient and induce production of NDP gene product.

In one embodiment, the NDP.AAV gene therapy construct may be administered intravenously. Intravenous administration is advantageous because it is the least invasive means of delivery the NDP.AAV construct. Intravenous administration may be performed in utero such that the construct is delivered to a foetus at the time the structures of the eye and ear are beginning to form. Intravenous delivery therefore allows for a straightforward means to achieve NDP gene therapy without the need for more complex procedures to be performed to a foetus. Intravenous administration may also be performed at any time after birth with minimum input from medical professionals. Intravenous administration can be performed in conjunction with other forms of NDP.AAV construct administration.

In one embodiment, the NDP.AAV gene therapy construct may be administered intraocularly. Intraocular injections using known techniques may be used to administer the NDP.AAV intraocularly. Intraocular administration is advantageous because it delivers the NDP.AAV directly to the site of retinal degeneration. Intraocular administration may include any one of intravitreal, subretinal, or suprachoroidal injections. Intraocular administration may be performed in utero and the use of in utero AAV injection for early gene expression has already been shown to be achievable in mice (Yasuda et al. 2021). Intraocular administration may also be performed at any time after birth. Intraocular administration can be performed in conjunction with other forms of NDP.AAV construct administration.

In one embodiment, the NDP.AAV gene therapy construct may be administered intracochlearly. Intracochlear injections using known techniques may be used to administer the NDP.AAV intracochlearly. Intraocular administration is advantageous because it delivers the NDP.AAV directly to the site of cochlear degeneration by preventing death of sensory hair calls and progressive hearing loss. Intracochlear administration may be performed in utero and the use of in utero AAV injection for early gene expression has already been shown to be achievable in mice (Chin-Ju et al. 2020). Intracochlear administration may also be performed at any time after birth. Intracochlear administration can be performed in conjunction with other forms of NDP.AAV construct administration.

In a further embodiment, NDP.AAV administration may be performed in any combination of intravenous administration, intraocular administration, and intracochlea administration. For example, administration of NDP.AAV may be by intravenous administration and intraocular administration or by intravenous administration and intracochlea administration or by intraocular administration and intracochlea administration or by intravenous administration, intraocular administration and intracochlea administration.

The use of NDP.AAV gene therapy provides for both early-stage and late-stage treatment to rescue vascular architecture in the ear and eye.

As described in the examples, early-stage treatment is crucial for full rescue of vascular pathology of the eye.

Embodiments are also envisaged in which NDP.AAV gene therapy may be used to increase activity of beta-catenin signalling in situations or ocular pathologies when other related pathways are affected (for example NDP LRP5 TSPAN5 & FRZ6) or when other components are mutated (for example TSPAN5). Further, as previously stated, NDP binds to a receptor complex, consisting of FZ-4, LRP-5/6 and TSPAN-12, to induce intracellular β-catenin signalling. In some embodiments, NDP.AAV gene therapy may be used to correct for pathology caused by mutations in other WNT signalling genes (for example FZD4, TSPAN12, LRP5/6).

In further embodiments, NDP.AAV gene therapy can be used to increases activity of beta-catenin signalling in situations or ocular pathologies when other related pathway genes are affected. As evidenced in the examples, the NDP.AAV gene therapy indicates efficacy of preventing vessel leakiness and, therefore, may be provided to Coates patients instead of steroids; even without mutations.

Treatment with NDP.AAV gene therapy may also benefit those suffering from progressive hearing loss with a vascular component, for example age related/and diabetic maculopathy and retinopathy, as well as retinopathy of prematurity (ROP), Familial exudative vitreoretinopathy (FEVR), and other NDP-related diseases. RNAseq data may be used to assess treatment outcomes. These include new druggable targets—e.g. Clu (or Fabp3)—Clusterin confers resistance to age related hearing loss—NDP.AAV will modulate CLU or other markers to protect against progressive or age related related hearing loss. Further, embodiments may include use of NDP.AAV gene therapy for to prevent exudation, retinal detachment, secondary glaucoma (causes pain) or phthisis bulbi (causing loss of the eye),

Certain embodiments relate to the sites of delivery needed for efficacy, such as fibrocytes of spiral ligament (SL), stria vascularis and lateral wall basal cell and marginal cells, glial cells of the modiolus; evidence for rescue after transduction of retinal muller cells, RGCs, RPE cells and PRS—RGC alone and Muller cells alone. This is evidenced by the discovery of the site of human NDP expression in the POM, fibrocytes and glial cells from human RNAseq analysis of human fetal cochlea.

As described in the example, it should be noted that signalling to hair cells is not required. Targeting in the ears is therefore not hair cells. This discovery provides the ability to properly target the causes of loss of hearing at different time points in development.

In one embodiment, the NDP.AAV is used in treatment of one or more of Norrie disease, age related hearing loss, diabetic maculopathy and retinopathy, retinal neovascularisation, retinal exudation, retinopathy of prematurity (ROP), Familial exudative vitreoretinopathy (FEVR), Coats disease, and other NDP-related diseases, or other beta catenin signalling-related retinal diseases by providing a first dose administered intraocularly neonatally. Delivering a first dose to neonates can rescue vascular architecture in the eye and vision. The rescue of vascular architecture includes deep layer vasculature. In one embodiment of the invention. the first dose is administered at about 14-17 postconceptual weeks (pcws), more preferably at about 15-16 pcws. In another embodiment, a second dose is administered intraocularly neonatally. A second dose may be given to a neonate in utero and may be beneficial for full vascularisation to occur. In one embodiment, the second dose is administered at about 22-26 postconceptual weeks (pcws), more preferably at about 24 pcws. In an alternative embodiment, a second dose is administered intraocularly at any time after birth. In a further alternative embodiment, a third dose may be administered intraocularly postnatally at any time after birth after receiving the first two doses as a neonate. Administration of the NDP.AAV intraocularly at later stages of development prevents retinal vessel leakiness. When applied at a late-stage vessel leakiness in retina is prevented which in turn prevents exudation and loss of the eye, phisis. As cell rescue can be achieved postnatally after administration intraocularly, it indicates that the effects are independent of vascular architecture pathology because this is completed in the neonatal stage. Administration of the intraocular doses can be performed using known techniques and may be an intravitreal injection.

In one embodiment, the NDP.AAV is used in treatment of one or more of Norrie disease, age related hearing loss, diabetic maculopathy and retinopathy, retinal neovascularisation, retinal exudation, retinopathy of prematurity (ROP), Familial exudative vitreoretinopathy (FEVR), Coats disease, and other NDP-related diseases, or other beta catenin signalling-related retinal diseases., wherein a first dose is administered intracochlearly neonatally. Delivering a first dose to neonates can rescue vascular architecture in the ear and prevents hearing loss. Death of sensory hair cells and progressive hearing loss is therefore prevented using intracochlearly administration. In one embodiment of the invention, the first dose is administered at about 13-20 postconceptual weeks (pcws), more preferably at about 15-18 pcws. In another embodiment, a second dose is administered intracochlearly neonatally. In one embodiment, a second dose is administered intracochlearly to an adolescent, more preferably at about 12 years old and below. In an alternative embodiment, a second dose is administered intracochlearly in adulthood, more preferably ages 12 years and above. In a further alternative embodiment, a third dose may be administered intracochlearly postnatally at any time after birth after receiving the first two doses as a neonate. The third dose may be administered intracochlearly at any time in adulthood, more preferably ages 12 years and above. Using the NDP.AAV of the present invention, death of sensory hair cells and progressive hearing loss can be prevented even when administered after development is complete. This means that treatment in life will be feasible.

With vessel barrier rescue in the cochlear and endocochlear potential rescue, sensory hair cells and hearing are protected without rescuing vascular morphology. The present invention therefore achieves long term amelioration of hearing loss.

In one embodiment, NDP.AAV may be used in treatment the of one or more of Norrie disease, age related hearing loss, diabetic maculopathy and retinopathy, retinal neovascularisation, retinal exudation, retinopathy of prematurity (ROP), Familial exudative vitreoretinopathy (FEVR), Coats disease, and other NDP-related diseases, or other beta catenin signalling-related retinal diseases., wherein a first dose of the medicament is administered by intravenously. In one embodiment, the first dose is administered neonatally or postnatally. In one embodiment, a second dose is administered neonatally or postnatally. In one embodiment, a third dose is administered neonatally or postnatally.

Further embodiments may require intravenous administration and/or intraocular administration, and/or intracochlear administration for more than three doses. For example, there may be 4, 5, 6, 7, 8 or more doses given to an individual. This administration may be prenatal or postnatal. Embodiments also include periodic administration by intravenous administration and/or intraocular administration, and/or intracochlear administration throughout the life of an individual.

In some embodiments, the construct may be specifically formulated for delivery of DNA or RNA molecules using non-viral vectors such as exosomes, nanoparticles or liposomes (e.g. lipofectamine) or viral vectors for example retroviral vectors, vaccinia virus vectors, adenoviral vectors, or herpes simplex viral vectors or lipid conjugated.

In the present invention, each construct can be packaged in any of the AAV serotypes. Construct 1 is the codon-optimised NDP open reading frame with the tags (SEQ ID NO: 19 (ITR to ITR sequence), SEQ ID NO: 20 (full plasmid sequence). Construct 6 is the codon-optimised open reading frame without the tags (SEQ ID NO: 21 (ITR to ITR sequence), SEQ ID NO: 22 (full plasmid sequence). For reference, the construct 0 (SEQ ID NO: 17 (ITR to ITR sequence), SEQ ID NO: 18 (full plasmid sequence) used in example 1 below and is in AAV2/9. SEQ ID NO: 23 is Construct 2 and is a construct comprises an NDP promoter. SEQ ID NO: 24 is Construct 3.

### EXAMPLES

The invention is further described in the following non-limiting examples.

### Example 1—A Study Demonstrating that Systemic AAV0.NDP Rescues Retinal Pathology and Hearing Loss in Model Norrie Disease

Norrie disease is a rare recessive X-linked dual sensory disorder, manifesting as congenital blindness and progressive hearing loss. It is caused by mutations in the NDP gene, which encodes norrin, a secreted Wnt-analog protein that induces canonical Wnt/β-catenin signalling through a FZ4/LRP5/6/TSPAN12 complex. Currently, no treatment for Norrie disease exists. Norrie vision loss results from early underdevelopment of the deep retinal vascular plexi during the late gestation, difficult to access with therapies. We have recently demonstrated that structural and barrier formation abnormalities in Ndp-KO mouse cochlear vasculature also start at early development and precede degeneration of the sensory outer hair cells and hearing loss, and thus may be causal to it via endocochlear potential reduction or metabolic stress. Importantly, people with Norrie have normal hearing in early childhood, and the onset of hearing loss often starts between teens to mid-twenties, suggesting that there may be a therapeutic window for intervention to prevent the degeneration of sensory cells and hearing loss. To investigate if gene therapy could be applied to treat Norrie disease, we created a tagged proof-of-concept norrin-expressing construct, allowing to study the distribution and detection of the transgenic norrin. The construct was demonstrated to produce the expected forms of protein and interact with its main receptor complex in functional assays despite the tags. Lastly, in vivo functionality of the construct in eye and ear was demonstrated in Ndp-KO mouse model.

This example of the present invention demonstrates the efficacy of an AAV9 vector, carrying a human NDP gene therapy construct delivered intravenously to the Ndp-KO mouse model at three clinically relevant stages of the disease progression. This example of the present invention shows that early postnatal treatment preserved both cochlear and retinal structure and function. Treatment of juvenile mice achieved full to partial rescue of cochlear structure and hearing function, but not the retina.

### Experimental Design and Function of the NDP Gene Therapy Construct In Vitro

To evaluate gene therapy in Ndp-KO mice we designed an experimental construct to express the human NDP gene. It was composed of the strong ubiquitous CAG promoter upstream of EGFP (to label the transduced cells), a self-cleaving P2A linker, full length human NDP coding sequence (402 bp including the native secretion signal) with a FLAG epitope sequence tagging the C terminus to aid detection of transgenic norrin, followed by a WPRE sequence and SV40 late poly A sequence at the 3′end (FIG. 1A). Construct expression and function were characterized in vitro in HEK293 cells. FIG. 1B′-B″ shows cytoplasmic EGFP in the transfected HEK293 cells that coincided with anti-FLAG immunostaining and labelled the cell surface (FIG. 1 B″, yellow). EGFP and NDP/norrin protein were detected in Western blots of transfected HEK293 cell lysates (FIG. 1C, FIG. 8). Recombinant NDP formed high molecular weight oligomers or aggregates (>250 kDa), which were reducible to NDP monomer-sized bands (16 kDa) (FIG. 1C, FIG. 8).

A TopFlash luciferase reporter assay in HEK293 cells was used to confirm the competence of the recombinant NDP to activate β-catenin signalling by interacting with its cognate receptor complex (FIG. 1D) (Chang et al 2015). The NDP expression construct induced luciferase activity when cotransfected with human FZ4, LRP6 and TSPAN12 expression plasmids (Chang et al 2015), but not alone (FIG. 1 E), consistent with the previously demonstrated NDP interactions with its receptor complex (Lai et al. 2017). Addition of lithium choride, known to stabilize β-catenin by inhibiting GSK3 (Zeilbeck, 2014) induced luciferase activity as expected without NDP or receptors. Together these data indicate the construct expresses biologically active recombinant NDP.

Safety and Transduction of Eye and Ear after Systemic Delivery of AAV9.NDP

To test expression of the NDP gene therapy construct in the mouse model, the AAV9 serotype was selected for packaging as it is capable of crossing the blood-brain barrier, but does not transduce vascular endothelial cells (Merkel et al. 2017). In other studies administering intravascular AAV9 achieved widespread transduction (Shibata et al. 2017; Massaro et al. 2020; Merkel et al. 2017). We predicted that use of a ubiquitous CAG promoter and AAV9 would provide delivery of the construct to the retina and cochlea after intravenous injection while avoiding direct damage of the eye and ear by local administration. Based on our previous observations the goal was to transduce cells close to the vessels in the vascularized sites of the cochlea—modiolus and the lateral wall—so that secreted NDP could target the endothelial cells of adjacent vessels and maintain the microenvironment conducive to sensory hair cell survival (FIG. 1 F).

To test treatment efficacy at timepoints relevant for people with Norrie disease progressive hearing loss and informed by our previous characterization of Ndp-KO mice (on the C57BL/6J) background (Bryant et al. 2022), three time points were chosen for vector administration, to represent different maturation and pathology stages in the eye and cochlea (FIG. 1 F): 1) neonatal (postnatal day P, 2); before onset of vision and hearing: at the beginning of retinal vasculature formation and before establishment of endocochlear potential; 2) juvenile—pre-degenerative (P21); eye vasculature and cochlea are recently matured; no hair cell death; 3) juvenile—degenerative (P30); onset of progressive hair cell death in the cochlea; neovascularization in the eye.

These correspond in developmental time to treatment delivery before birth, in children and young adults. FIG. 1F summarizes the study design. AAV.GFP was delivered by intravenous injection to groups of neonatal mice (2.73E+13 vg/kg; dose P2-L), juvenile mice at P21 at 2 doses (5.45E+12 vg/kg; dose P21-L and 2.74E+13 vg/kg; P2-H) and juvenile at P30 (1.37E+13 vg/kg; dose P30-H). Treated mice were monitored periodically and showed normal weight and general health compared to controls Control samples were pooled in WT and Ndp-KO groups as no differences were observed in readouts of control mice, injected with PBS at any time point (FIG. 2 A, FIG. 9).

At 2 months of age, transduction of retina and cochlea was confirmed by GFP immunostaining (FIG. 2). P2-injected retinas were most efficiently transduced in the central area (Suppl Fig Y), which is vascularized at P2 (FIG. 2B, FIG. 10). P21 administration resulted in widespread transduction of the retina (FIG. 2 C), consistent with the full coverage of the inner retinal surface with the vasculature in Ndp-KO (FIG. 10). Retinal ganglion cells were efficiently transduced in early or late treated mice was, and expression in Muller glial cells, a known site of Ndp expression (Ye et al. 2009) was rare (FIG. 2 B, C).

In the cochlea, transduction was achieved in the modiolus and lateral wall proximal to putative targets of NDP signalling, which are the lateral wall vasculature and the hair cells (Hayashi et al. 2021; Rehm et al. 2002) (FIG. 2 F). Spiral ganglion neurons were transduced in all treatment groups (Fig S4), and fibrocyte-shaped cells in the lateral wall (potentially type II and IV fibrocytes) and modiolus (FIG. 2F-I). Inner hair cells were transduced sparsely and only in P2-L group; no transduction observed in the outer hair cells, vascular endothelial cells, pericytes, satellite glia cells.

In summary, transduction of both neonatal and juvenile cochlea and retina was achieved by the AAV.NDP vector with lower levels in juveniles compared to neonatal administration. Vascular cells were not transduced.

Neonatal Treatment with AAV9.NDP Rescues Retinal Vasculature and Vision

We assessed the effect of AAV NDP gene delivery on the retinal pathology by analysing retinal vasculature (FIG. 3 A,B) in the eyes of mice at 2 months of age after treatment at P2-L and P21-H using retinal cryosections and whole mounts (Previous studies in Ndp-KO mice showed that development of the superficial retinal vasculature is slower than normal, but complete by P20, and the deep layer vasculature never forms (Richter et al. 1998; Luhmann et al. 2005a). FIG. 10 shows the appearance of Norrie retina at the treatment time points.

Vasculature in the three vascular plexi in observed in retinal wholemounts of the WT (FIG. 3 C) and only the superficial plexus in those of the Ndp-KO (FIG. 3 D). Treatment at P2, but not at P21, rescued all the three plexi (FIG. 3 E, F). Vascular network formation in the plexi was confirmed in color-coded Z-stack depth projections of retinal whole mounts (FIG. 3 C-F). Three vascular networks at different depths were also detected in cryosections of WT and P2-treated mice (FIG. 3 G, I), but in Ndp-KO and P21-H, an abnormally neovascularized plexus was present on the superficial retina only, and individual, non-branching neovascular tufts (FIG. 3 H, J arrows). The blood-retinal barrier is normally established by P17-P20 (Fruttiger 2002). By 2 months immunostaining of the Ndp-KO retina showed reduced expression of claudin-5, a structural component of endothelial cell tight junctions and increased expression of PLVAP a structural component of endothelial fenestrae, compared to WT, previously reported as early markers of the abnormal Norrie retinal vasculature (Wang et al. 2012). In both the P2-L and P21-H groups, expression of claudin-5 was restored and PLVAP staining, typical to Ndp-KO, disappeared (FIG. 3 K-N), consistent rescue of the vascular phenotype.

To assess the effect of AAV NDP delivery on visual function, scotopic electroretinograms (ERG) were recorded from the P2 and P21 treatment groups and controls at 1.5 months of age. FIG. 3 O represents the typical ERG scotopic traces of WT, Ndp-KO, P2-L and P21-H groups in response to a bright 10000 mcd/s\hat{ }-2 flash. The pronounced b-wave in the WT, signifying signal transduction from photoreceptors to bipolar cells, was almost flat in Ndp-KO (FIG. 3 O). P2-L treated animals resembled the WT and showed some recovery of the oscillatory potentials, although the amplitude did not reach its full height, whereas P21-H ERG trace was similar to Ndp-KO (FIG. 3 O). Suppl FIG. 12 demonstrates the full set of average traces for each group (FIG. 12 A) and the ratio of a-wave to b-wave amplitudes (FIG. 12 B,C). There was an improvement of oscillatory potential amplitudes was observed, though it did not reach significance in either group (FIG. 3 P)

There were no differences in the a-wave parameters between WT and Ndp-KO, nor the treatment groups (FIG. 12 D-E). b-wave amplitudes between WT and Ndp-KO were significantly different with large effect size (FIG. 3 Q, R). P2-L b-wave showed a significant improvement, consistent with the revascularization of the deep retina (FIG. 3 Q). In P21-H, b-wave amplitude was partially restored in the highest intensity flash responses (FIG. 3 R) In summary these data indicated efficacy of intravenous delivery of the AAV.NDP vector to improve the retinal pathology. Treatment prior to retinal vasculature maturation but not at later time points rescued deep retinal vascular pathology. In previous studies using genetically engineered mice restoration of the deep vascular plexi in Norrie was not possible after the maturation point (P17-20) (Wang et al. 2012).

### Norrie Disease Biomarkers in the Cochlea Respond to AAV.NDP Treatment

As understanding of the downstream molecular mechanisms leading to the cochlear insults in Norrie disease is limited and to provide biomarkers to assess treatment efficacy in the cochlea, we compared the patterns of gene expression in the WT and Ndp-KO cochlea and in mice treated at P2. NDP downstream targets in adult mouse cochlea and Norrie pathology-associated transcription has not been analysed before. Dysregulated gene expression profiles were identified by RNAseq analysis of whole-cochlea from male WT (n=4), Ndp-KO (n=3) and P2-L (n=4) mice at 2 months.

Differential gene expression analysis identified 45 genes significantly differentially expressed (adjusted p≤=0.05) between WT and Ndp-KO cochleas and 35 genes significantly differentially expressed between Ndp-KO and treated Ndp-KO P2-L samples (FIG. 13). There were no genes significantly differentially expressed between the WT and Ndp-KO P2L groups indicating rescue with treatment. Among the 45 and 35 significantly differentially expressed genes (DEGs), there was an overlap of 16 genes indicating rescue to normal expression levels. Also, unsupervised clustering of all samples based on the expression of the 45gene s differentially expressed between WT and Ndp-KO resulted in the treated Ndp-KO P2-L samples clustering with the WT samples rather than the untreated Ndp-KO samples (FIG. 4.) Slc7a1, Flt1, Abcb1a, Cldn5 were downregulated in the Ndp-KO and returned to normal levels with treatment (FIG. 4A). They are likely downstream targets of NDP signalling, associated with the normal function of cochlear microvasculature: barrier (Cldn5), pericyte-driven vascular branching and barrier (Flt1) (Eilken et al. 2017; Wang et al. 2019; Zhang et al. 2021), transport of molecules (Abcb1a, Slc7a1). Abcb1a is associated with hearing loss and increased sensitivity to ototoxicity in mice (Li et al. 2019; Zhang et al. 2000). Slc7a1 is an amino acid transporter, typical to normal BBB (Yahyaoui and Perez-Frias 2019). The set of genes upregulated in the Ndp-KO, by their function, could be related to the stress response in the cochlear pathology (FIG. 4 A).

These findings were consistent with microvasculature as a primary site of pathology. Notably genes expressed in hair cells were not found to be differentially expressed even though marked hair cell loss is apparent by 2 months (Bryant et al 2022). Dysregulation of barrier markers and transporters, supports the hypothesis that microvascular disruption leads to an unsuitable microenvironment for hair cell survival in Norrie cochlea.

Expression of a set of the newly identified biomarkers of pathology were analysed by qRT PCR after treatment at later time points, together with analysis of the levels of transgenic NDP GFP expression by qPCR and pericellular permeability gene Plvap, which we previously showed was dysregulated in Ndp-KO cochlea at 2 months (Bryant et al. 2022).

Levels of transgene NDP GFP expression in the cochlea were highest after treatment in neonates. In juveniles, expression corresponded to dosage with higher expression at P21 and P30 compared with the low dose treatment at P21 (FIG. 4 B) These patterns are in line with the patterns of GFP transduction observed in whole mount cochlea whole mounts (FIG. 11) indicating dependence of transduction levels on age (see P2-L and P21-H groups, injected with the same amount of vg/kg).

At 2 months gene expression returned to WT expression levels in P2-L and P21-H groups, and at least partially/significantly improved in P30-H samples, while P21-L group was not significantly improved (FIG. 4 B-E). Two-way ANOVA with Tukey's post hoc test showed that Cldn5 was downregulated in Ndp-KO, but restored completely in P2-L group. From the treatments of juvenile mice only the higher dose at P21 (P21-H) showed Cldn5 levels similar to WT (FIG. 4). Plvap demonstrated its high sensitivity to AAV NDP treatment even at lowest doses: the increased expression of Plvap was successfully downregulated to WT levels in all treatment groups (FIG. 13). The rescue of both markers at P21 suggests that even mature Norrie cochlear vasculature is still responsive to pericellular tight junction barrier restoration.

Together these data indicated that dysregulated gene expression levels were restored to those of wild type not only after neonatal treatment but also after later treatment of juvenile mice. As the amelioration of expression of the selected hallmarks of the molecular micro vasculature pathology suggested that delivery of NDP by gene therapy is maintaining barrier and transport function.

Effect of AAV.NDP on the Lateral Wall Vasculature and Organ of Corti Sensory Hair Cell Survival after Treatment of Neonates and Juveniles

To assess whether the biomarker gene expression patterns correspond with rescue of tissue pathology, cochlear whole mounts from AAV.NDP treated mice were analysed by immunostaining to assess the effect on lateral wall microvasculature and survival of hair cells in the organ of Corti. As previously we identified malformation of cochlea microvasculature to be an early lesion site (Bryant et al. 2022), we compared the effects of AAV NDP gene therapy on the lateral wall vasculature morphology (FIG. 5 A) after treatment of neonatal and juvenile mice by endomucin immunostaining. Quantification of branching points in stria vascularis vessels {and spiral ligament}along the lateral wall demonstrated the reduced branching in the Ndp-KO, which was most marked in the apical region (region 1/8 and 2/8 in apex-to-base axis) in the Ndp-KO had a significantly reduced branching compared to the WT (analysed with repeated measures two-way ANOVA with Dunnett's post hoc test, P≤ . . . ) (FIG. 5B, C-F).

In the spiral ligament capillary network, we found treatment of neonates, but not at later timepoints also improved malformation. There was an even distribution endomucin and claudin-5 staining on the WT vessels (FIG. 5. G-G″) whereas Ndp-KO (FIG. 5 H-H″) showed most vessels with low/absent claudin-5 and some atypical vessels with high claudin-5 coincident with abnormally low endomucin as previously described.

In P2-L treatment group, vascular network appearance was comparable to WT, with even distribution of endomucin and claudin-5 staining (FIG. 5.1-I′), signifying a good rescue. In P30-H treatment groups, vessel diversification was noticeable: part of the vasculature was forming meshworks with atypical appearance similar to Ndp-KO were observed (FIG. 5 J-J′), suggesting that the rescue was not achieved. These data suggests that NDP is required for the early development of the strial apical tip which is still forming at P2, and this pathology is irreversible by later treatment after complete stria maturation (P20) (Ando and Takeuchi 1998).

AAV.NDP Prevents OHC Loss Even after the Onset of Degeneration

We analysed levels of hair cell survival at 2 months of age compared with WT and untreated Ndp-KO mice. At 2 months, hair cells in the WT cochlea were almost intact (FIG. 6 A-A″), while Ndp-KO had a severe degeneration of OHCs in the mid frequency region in regions 2-4 out of 8 corresponding to 6-18 kHz (FIG. 6 B-B″) consistent with our previous study (Bryant et al. 2022). In the treatment groups OHCs were either preserved entirely, as in groups P2-L and P21-H (FIG. 6 C-C″, D-D″″) or partially P30-H (FIG. 6E-E″). The surviving OHCs were quantified (FIG. 6 F-K) in the whole mounts of the organ of Corti, mapped into equal distance regions along the apex-to-base axis. Data was analysed with two-way repeated measures ANOVA with Dunnett's post hoc test for each treatment group individually, comparing the corresponding regions with the WT (FIG. 6 F-K). Analysis confirmed a complete OHC rescue in P2-L and P21-H (FIG. 5 I, K) groups and significant improvement in the “sensitive” region ( 2/8- 4/8 from the apex) of P21-L and P30-H samples (FIG. 5 J, L). FIG. 14 shows hair cell survival along the apex-base axis of the Organ of Corti in all groups The cochlear whole mount analyses indicated that treatment of neonates improved microvasculature and prevented onset of hair cell degeneration and importantly later treatment of juveniles also reduced progressive hair cell degeneration.

### AAV9.NDP Treatment of Ndp-KO Juvenile Mice Rescues the Auditory Function {Mention Advanced Stages}

Finally, to evaluate the therapeutic effect on hearing we performed audiological and electrophysiological assessment of the cochlea function at 3 months of age (FIG. 7). Endocochlear potential was significantly reduced in Ndp-KO compared to WT, but recovered in the treated mice after treatment of neonates and juvenile mice (FIG. 7A).

To estimate the functionality of hair cells, we analysed DPOAE thresholds at 6-30 kHz. The overlay of threshold averages is shown in FIG. 7 B, and statistical analysis in FIG. 15. Thresholds in the mid frequency regions at 6-18 kHz, were significantly elevated in the Ndp-KO compared to the WT, consistent with loss of hair cell integrity at 2 months in these regions of the organ of Corti (FIG. 6) and with our previous analysis (Bryant et al., 2022). Unexpectedly, in the higher frequency regions where little hair cell degeneration was observed in the Ndp-KO, WT thresholds were elevated and showed significantly worse function than Ndp-KO at 30 kHz (FIG. 7 B, arrow). This likely reflects rapid onset of age-related hearing in these WT control mice, which is a known feature of C57BL/6 mice at later stages (Johnson et al., 1997). For completeness we have included auditory and electrophysiological analyses at all frequencies and marked the “crossing point” in the frequency range at which the WT controls display the paradoxical auditory dysfunction.

Thresholds of Ndp-KO in the mid frequency regions at 6-18 kHz, were significantly elevated in the Ndp-KO compared to the WT but were fully restored after treatment of neonates (P2-L). Treated juveniles also showing function indistinguishable from that of the WT at 12-18 kHz (FIG. 7 B-B′″) consistent with the survival of hair cells observed in the organ of Corti after treatment.

At the highest frequency tested (30 kHz) the treated mice and the untreated Ndp-KO mice both showed DPOAE thresholds significantly lower that of the WT (FIG. 7 B-B′″)

To evaluate the restoration of hearing, we recorded auditory brainstem responses (ABR) to broadband (click) and pure tone stimulus. The click thresholds were significantly increased in Ndp-KO in comparison to WT but returned to WT values in all treatment groups (FIG. 7 C), indicating that a good overall rescue of hearing was achieved.

We next analysed the frequency specific ABRs to pure tone stimuli at 3-42 kHz. The Ndp-KO thresholds were elevated at 3-18 KHz frequencies compared to WT. In line with the DPOAE analysis above 24 kHz, thresholds of the WT were increased, while Ndp-KO maintained hearing within normal limits (?REF) (FIG. 7 D-D′″, FIG. 15). After AAV.NDP treatment of neonates and juvenile mice, thresholds at the 30-18 Hz frequencies were reduced, with complete restoration to WT thresholds achieved only with neonatal treatment.

In the high frequency region (30-42 kHz), the Ndp-KO had better hearing than the WT, both P21-H and P30-H displayed even better hearing than the Ndp-KO (FIG. 7 D″-D′″), whereas the P2-L group average thresholds indicated a slightly worse hearing than that of the Ndp-KO (FIG. 7 D′). check this difference is significant

In summary, these EP, DPOAE and ABR results indicate, that hearing function can be preserved by AAV9.NDP gene therapy after treatment at a range of disease stages. Considered together these data suggest rescue is likely mediated via prevention of progressive hair cell loss and preservation of the OHC function, as the affected and rescued tonotopic regions co-incided in the DPOAE and tone ABR readouts, and the OHC degeneration and rescue regions (FIG. 7 E).

### Discussion

AAV.NDP Treatment Prevents Disease Progression and, Responsiveness and Time of Treatment

This proof-of-concept study has investigated the possibility of gene replacement therapy application to treat Norrie disease. We have for the first time demonstrated that early treatment provides complete rescue of cochlear pathology and retinal vasculature proving that our vector is delivering functional human Norrin. The early treatment time point corresponds to fetal stage in human development. Fetal gene therapy is in its infancy and in the case of NDP, may risk adverse effects on placental vasculature or the developing fetus (Ye et al. 2009). However, we also show that the Norrie cochlea is responsive to treatment across different stages of the disease, including after the onset of degenerative changes. These changes correspond to childhood or young adulthood in Norrie patients, after sensory system development is complete. This suggests that treatment of patients may be both feasible and deliverable. The low cell turnover of cochlea suggests that NDP.AAV gene replacement therapy could enable long-term provision of norrin to maintain cochlear vessel barrier function through life.

In the eye, lack of Norrin results in a failure to develop the two deep layers of retinal vasculature. Vision loss in Norrie disease is usually present from birth (Redmond et al. 1993). Our work has demonstrated that although the missing deep retinal vasculature cannot be regrown with NDP after the vascular network maturation, consistent with study by others (Wang et al. 2012), the retinal vascular barrier responds to postnatal AAV NDP and remains responsive to treatment over time. This offers a route to treat NDP associated ocular conditions in patients.

### The Role of Vasculature in the Norrie Cochlear Phenotype

The responsiveness of the cochlea to treatment at late timepoints is consistent with the pathology being mediated by vascular barrier dysfunction. Lack of NDP does not result in major morphological abnormalities or absence of the cochlear vasculature but only causes a disruption of the blood-labyrinth barrier (Bryant et al. 2022), Blood-labyrinth barrier maintenance is essential for maintenance of the endocochlear potential and, through that, for survival and function of the hair cells, (Liu et al. 2016). RNAseq and qPCR analysis of whole cochlea lysates demonstrated that there was a pronounced dysregulation of vascular barrier and transport factors and subsequent restoration after AAV-NDP treatment at early and late timepoints. Moreover, downregulated expression of transporters Abcb1a, Slc7a1 could be causing metabolic stress. Restoration of the vascular gene expression, but not the cochlear vascular morphological defects coincided with the rescue endocochlear potential, OHCs and hearing. The apparent reversibility of the cochlear vasculature barrier even as disease progresses therefore allows restoration of the normal hair cell environment and their normal function.

It has been suggested that in the cochlea norrin may act directly on the HCs in regulating maturation and gene expression via the transcription factor Pou4f3, (Hayashi et al. 2021). Both constitutive NDP overexpression in supporting cells or neonatal b-catenin stabilization in hair cells using Atoh1-Cre was reported to preserve OHC survival (Hayashi et al. 2021). In our study we did not detect a reduction in Pou4f3, or hair cell gene expression (e.g. Myo7a), in RNAseq analysis at 2 months of age, despite the observed loss of OHC in Ndp-KO cochlea, indicating the resolution of bulk RNAseq analysis. Our study specifically shows that OHCs, which differentiated and matured in the absence of Ndp were able to survive (FIG. 6) and function (FIG. 7, DPOAEs) long term if NDP was restored at P21 or 1 month of age.

### Developing Clinical AAV.NDP Gene Therapy and Safety

Gene therapy is a quickly moving field. Although a majority of treatments are at the pre-clinical trial stage, three AAV-mediated therapies—Luxturna®, Zolgensma® and Glybera®—have been approved to treat severe genetic disorders. Secreted signalling protein gene therapy is not yet widely studied, and there are potential side-effects of unregulated prolonged expression. The viral dose used to achieve rescue of the cochlea in this study, was low, approximately 5-25 times lower than that of clinically approved Zolgensma® for the treatment of the life-limiting condition spinal muscular atrophy which used a similar AAV9 vector (1.1×10¹⁴ vg/kg). Rescue via our ubiquitously expressed NDP construct implies that targeting specific cells is not necessary as NDP is secreted and reaches the necessary target cells. This is consistent with rescue achieved in previous reports via ectopic overexpression of NDP in transgenic mice (Ohlmann et al. 2005; Bassett et al. 2016). The AAV.NDP gene therapy construct transduced cells in the proximity of the sites of cochlear pathology but largely did not transduce the affected vasculature endothelial or sensory hair cells.

As systemic delivery of AAV can cause side effects, direct delivery to the eye and ear may be more suitable for clinical application. Complete longitudinal toxicology studies are needed to establish the safety of AAV.NDP gene therapy. This proof-of-concept study demonstrates for the first time that Norrie disease pathology responds to gene replacement therapy and opens the way for targeted gene delivery to treat progressive hearing loss and leaky retinal vessels. Such NDP gene delivery may be useful in alleviating ocular disease in milder, FEVR-like cases of Norrie (Wawrzynski et al., 2022) or in combination with planned preterm delivery in cases of prenatal diagnosis of Norrie (Sisk et al. 2014). AAV.NDP gene replacement may also have potential for treatment of peripheral vascular disease symptoms in Norrie patients. To our best knowledge, this is the first such application of systemic AAV9 delivery to treat a progressive cochlear disorder (Shibata et al. 2017).

### Methods

Gene expression plasmids: The CAG>EGFP-P2A-NDP-FLAG pAAV gene therapy construct was designed using vectorbuilder.com and purchased from Cyagen as an E. coli stock. Plasmids expressing human FZ4, LRP6 and TSPAN12 plasmids (Chang et al. 2015), were provided by Prof Yvonne Jones (Oxford). M50 Super 8× TopFlash in pTA-Luc vector (12456) M51 Super 8× FopFlash in pGL3 vector (12457) were obtained from Addgene. All plasmids were expanded using standard methods and purified using the Miraprep protocol (Pronobis et al. 2016). HEK293 cells were cultivated in regular 5% CO₂ cell culture incubators at 37° C., in DMEM-high glucose medium (11965-084, Gibco) with 10% FBS (A38401, Gibco) (5% CO₂, 37°). Cells were passaged at 1:5 ratio using 1× trypsin/EDTA (25300-054, Gibco).

TopFlash assay.: EK293 cells were plated at equal densities in 96 well plates. The next day cells were transfected (Transfection mix: 40 μg of TopFlash plasmid, 10 μg of mCherry, and a combination of 10 g of each of the NDP gene therapy construct and plasmids Norrin receptors) using FuGENE® HD reagent (21 FuGENE: lg DNA) for 24 h followed by washing and replacement with regular tissue culture medium. 5 mM LiCl treatment for 24 h was used as a positive control for β-catenin activation. Following that, cells were assayed for β-catenin activity using Dual-Luciferase® Reporter Assay System (E1910, Promega), according to manufacturer's instructions. Induced luminescence was measured with luminometer Fluostar Optima, (BMG Labtech), using recommended readout parameters.

Western blotting: HEK293 cells were transfected with the NDP gene therapy construct as described above. 48 h after the removal of transfection medium, cells were harvested in RIPA buffer containing protease inhibitor cocktail cOmplete™ Mini (11836153001, Promega). Total protein was extracted and quantified by Bradford assay according to standard methods. Then samples were diluted mixed with 4× Laemmli sample buffer (BioRad) with or without 5% β-mercaptoethanol, and heat inactivated at 75° C. or incubated at room temperature for 10 min, then maintained on ice. 20-30 g of protein was loaded per well on a 1 mm 12% SDS-PAGE gels and separated by electrophoresis (Mini-PROTEAN, BioRad) followed by transferred on 0.2 μm pore size nitrocellulose membrane (BioRad) in TransBlot semi-dry transfer system. Membranes were washed in PBST, blocked in 5% nonfat milk (Blotting-Grade Blocker, BioRad) in PBST and probed using relevant antibodies: Anti-GAPDH EMD Millipore MAB374, Anti-Actin, Anti-FLAG eBioscience 14-6681-80, Anti-GFP Abcam Ab6662, Anti-Desmin.

RNA extraction: Cochleas were isolated from surrounding tissue and the vestibular and snap frozen. Retinas were dissected from the eye in cold PBS and snap frozen. Total RNA was extracted using a modification of a published protocol (Vikhe Patil et al. 2015) (TRI Reagent® 93289-25ML Sigma-Aldrich, DirectZol kit). RNA was eluted in 40-50 ul of nuclease-free water and analysed using the by NanoDropm 2000 (Thermo Scientific) and Agilent Bioanalyzer platforms.

Gene expression analysis: cDNA was synthetised from 100 ng RNA using RvertAid H Minus First Strand cDNA Synthesis kit (K1631) with random hexamers according to manufacturer's instructions. cDNA equivalent to 1 ng of RNA per reaction was used for gene expression analysis with PowerSYBR® Green PCR Master mix (436759) and relevant primers (Cldn5: F:5′ TTAAGGCACGGGTAGCACTCACG3′ (SEQ ID NO:5), R:5′ TTAGACATAGTTCTTCTTGTCGT3′(SEQ ID NO: 6), Plvap: F5′ GTGGTTGGACTATCTGCCTC3′ (SEQ ID NO: 7), R:5′ATAGCGGCGATGAAGCGA3′ (SEQ ID NO: 8), Actin-b: F5′ TGTTACCAACTGGGACGACA3′ (SEQ ID NO: 9), R:5′ CTGGGTCATCTTTCACGGT3′ (SEQ ID NO 10), Abcb1a, Slc7a1, Flt1). Whole transcriptome analysis was done using strand specific RNA seq with poly-A selection on the Illumina Nova-seq, with a target library size of 20 million paired end 150 bp reads per sample. Virus production and packaging: The gene therapy construct was packaged into AAV capsids in UCL AAV Facility, using HEK293T/(AAV?) cell culture, as described previously. Virus titre was determined by RT-PCR using the linearized construct plasmid as a standard (ITR F:5′ GGAACCCCTAGTGATGGAGTT3′ (SEQ ID NO: 11), R: 5′CGGCCTCAGTGAGCGA3′ (SEQ ID NO: 12), EGFP F:5′ AGTCCGCCCTGAGCAAAGA3′ (SEQ ID NO: 13), R:5′ TCCAGCAGGACCATGTGATC3′ (SEQ ID NO: 14)

### Animal Experiments.

Animal studies were carried out after University College London and King's College London Ethics Review and in accordance with UK Home Office regulations and the UK Animals (Scientific Procedures) Act of 1986 under UK Home Office license. Mice were kept at 12 hours light, 12 hours dark cycle and provided food and water ad libitum Mice carrying the Ndp^{Tm/wbrg} (Ndp) allele were provided by Prof W. Berger ((Berger et al. 1996) and the line was maintained by crossing heterozygous Ndp^{+/-} females with WT C57BL/6 males from Charles River. Ndp^{v/-} males and Ndp^{-/-} females (Ndp-KO) and littermate or age matched age Ndp^{v/+} males or Ndp^{+/+} females (WT) were used as in experiments. Ndp^{-/-} females are known to be infertile (Luhmann et al. 2005b). Genomic DNA was isolated from from ear or tail biopsies and genotypes determined by PCR (MyFi Mix (BIO-25050); F: 5′ GTATTGCATCCATATTTCTTGG3′ (SEQ ID NO: 15) R: 5′CTCTCCATCCCCTGACAAGGA3′ (SEQ ID NO: 16), WT amplicon=528 bp, KO amplicon ˜1500 bp)

Treatment with the AAV construct. 8 μl of AAV construct in PBS carrying . . . vg (“low”) dose was injected in the superficial temporal vein of neonatal mouse pups (P2) using a 100 μl Hamilton syringe. Mice at P21 or P30 before injection were maintained at at 38° C. and kept for 10 min to dilate the vasculature. 40 μl or 50 μl of AAV construct, carrying either . . . vg (“low”) or . . . vg (“high”) dose was injected into the tail vein. Littermate controls were injected with a matched volume of PBS. Treated mice were monitored and weighed 3 times a week until P30, then once a week.

Audiology (ABR, DPOAE, EP) were performed as described previously described (JCI, ourselves)

Electroretinograms Electroretinograms were performed similar to published protocols Ohlmann et al (45). Mice were dark-adapted overnight for a minimum of 12 hours, anaesthetised by isoflurane inhalation and their pupils were dilated (1% tropicamide eye drops) and anaesthetised (proxymetacaine eye drops). The mouse was then connected to the OcuScience® HMsERG system according to the manufacturer's instructions. Single flash recordings were obtained under dark adapted (scotopic) conditions using stimulus intensities 0.1 mcds/m², 1 mcds/m², 3 mcds/m², 10 mcds/m², 30 mcds/m², 0.1 cds/m², 0.3 cds/m², 1 cds/m², 10 cds/m², and 25 cds/m². Ten responses were averaged at each intensity.

TRITC-BSA permeability assay. Mice at 2 months of age were injected with 2.5% TRITC-BSA (Sigma) in PBS (w/v) into the tail vein. Mice were sacrificed by cervical dislocation, after 45 min. Tissue samples were fixed and immunostained as described below.

Tissue processing: Eyes were isolated and fixed in 4% paraformaldehyde (PFA) for 60-90 min followed by multiple washes with PBS. Cochleas were isolated, dissected out of the auditory bulla. The cochlear apex and oval and round windows opened and 1 ml 4% paraformaldehyde (PFA) injected through the round window. Fixation was continued in 4% PFA for 90-120 mins, followed by decalcification in 4% EDTA in PBS (W/v), pH 7.4, for 72 h and multiple washes in PBS. Brains Right hemisphere was carefully removed from the skull and fixed for 24 h in 4% PFA, then washed 3 times for 10 min in PBS.

Histology: Eyes were enucleated, then sequentially equilibrated in 15% and 30% sucrose, embedded in OCT medium (Thermofisher), snap-frozen in a dry-ice isopentane slurry and stored at −80° C. Sections were cut at 12 μm thickness on a Leica cryostat and mounted on SuperFrost Plus glass slides (Thermofisher). Cochleas were embedded in 4% low melting grade agarose in PBS. 150-200 μm thick Cross-sections were cut using a vibratome and stored in PBS at 4° C. until further processing. Retinal wholemount preparations were made by removing the sclera, choroid and RPE from the posterior segment of the eye and making 5 radial incisions into the retina with the longest incision marking the ventral retina. Cochlear wholemount preparations were made by removing the otic capsule and separating the lateral wall and modiolus by cutting beneath the spiral prominence/stria vascularis.

Immunohistochemistry: Tissue Samples were incubated in permeabilization/blocking solution (5% FBP, 1% BSA in PBS containing 0.1% (tissue sections) or 0.5% (wholemounts) Triton X-100) Samples were incubated with primary antibodies diluted in permeabilization/blocking solution overnight at 4° C., washed with PBS, incubated 2 h secondary antibodies at room temperature, washed with PBS and mounted with Prolong Diamond (P36970, Invitrogen). Primary antibodies: Anti-Endomucin (SantaCruz Sc53941, 1:100), Anti-Desmin (Proteintech 16520-1-AP, 1:300), Anti-Myo7a (Proteus 25-6790, 1:200), Anti-Claudin5 (Invitrogen 34-1600, 1:500), Anti-FLAG (Invitrogen 14-6681-80,), Anti-NDP (R&D systems AF3014), Alexa-fluor 594 anti-tubulino (BioLegend, 801207 1:500) FITC anti-GFP (Abcam Ab6662), Alexa-fluor 488 anti-GFP (Invitrogen A21311). Secondary antibodies: Anti-mouse IgG(H+L) Alexa Fluor 488 (Life Technologies A11001 1:500), Anti-mouse IgG(H+L) Alexa Fluor 594(Life Technologies A21203 1:500), Anti-rat IgG(H+L) Alexa Fluor 647 (ThermoFisher A21247 1:250), Anti-rabbit IgG(H+L) Alexa Fluor 488 (Life Technologies A21206 1:250), Anti-rabbit IgG(H+L) Alexa Fluor 568 (ThermoFisher A11036 1:250). Markers: Alexa Fluor 647 phalloidin conjugate (Life Technologies A22287 1:200), Alexa Fluor™ Plus 750 phalloidin conjugate (Invitrogen A30105, ?), Alexa Fluor 594 isolectin GS-IB4 conjugate (Life Technologies 121413, 1:100), Alexa Fluor 647 isolectin GS-IB4 conjugate (Invitrogen 132450, ?) Samples were imaged using Zeiss Observer, . . . spinning disk or Olympus microscopes.

Branch point analysis: Branch points of strial capillaries were manually quantified from low magnification images of lateral wall wholemount preparations using ImageJ. Vascular “intersection” points, which had 3 branches connected, were considered branch points.

Hair cell quantification was performed on low magnification images of wholemount preparations of the organ of Corti. Each organ of Corti samples was mapped using the Measure_line macro for ImageJ (REF, Liberman) and divided into 24 equal pieces. Using a custom-made ImageJ macro, 200 μm long rectangular were sampled from each piece and MyoVIIA-positive hair cells counted using local maxima detection. Empty slots left by dead cells were counted manually. Percentage surviving cells were calculated as surviving/(dead+surviving)*100%. Values from 3 adjacent regions were averaged giving a total of 8 regions per organ of Corti. Data from treated and control groups was analysed using 2-way ANOVA and Dunnett's post hoc tests for multiple comparisons (GraphPad PRISM v7.0).

| | | | Catalog | | |
|---|---|---|---|---|---|
| | Host | | number | | |
| Antibody | species | Type | (RRID) | Company | Dilution |
| Anti-Endomucin | rat | monoclonal | Sc53941 | SantaCruz | 1:100 |
| | | IgG1 | (AB_2100038) | | |
| Anti-Nf200 | chicken | polyclonal | Ab4680 | Abcam | |
| Anti-Desmin | rabbit | polyclonal | 16520-1-AP | Proteintech | 1:300 |
| | | | (AB_2292918) | | |
| Anty-Myo7a | rabbit | polyclonal | 25-6790 | Proteus | 1:200 |
| | | | (AB_10015251) | | (1:100 |
| | | | | | for |
| | | | | | 3D?) |
| Anti-Neurofilament | rabbit | polyclonal | N4142 | Sigma | [symbol] |
| 200 (NF200) | | | (AB_477272) | | 1:1000? |
| Anti-Plvap | rat | monoclonal | 553849 | BD | 1:100? |
| | | | | Biosciences | |
| Anti-Claudin5 | rabbit | polyclonal | 34-1600 | Invitrogen | 1:500? |
| Anti-FLAG | mouse | Monoclonal | 14-6681-80 | Invitrogen | |
| Anti-NDP | goat | polyclonal | AF3014 | R&D | |
| | | | | Systems | |
| Conjugated | | | Catalog | | |
| primary | | | number | | |
| antibodies | Host | Type | (RRID) | Company | Dilution |
| Alexa-fluor 594 | mouse | monoclonal | 801207 | BioLegend | |
| anti- tubulin ß3 | | | | | |
| Anti-GFP-FITC | goat | polyclonal | Ab6662 | Abcam | |
| Anti-GFP-488 | rabbit | polyclonal | A21311 | Invitrogen | |
| Alexa Fluor TM 488 | | | | | |
| | | | Catalog | | |
| Target/ | | | number | | |
| modification | Type/IgG | Host | (RRID) | Company | Dilution |
| Alexa Fluor 647 | — | — | A22287 | Life | 1:200 |
| phalloidin | | | (AB_2620155) | Technologies | |
| conjugate | | | | | |
| Alexa Fluor TM | — | — | A30105 | Invitrogen | |
| Plus 750 phalloidin | | | | | |
| conjugate | | | | | |
| Alexa Fluor 594 | — | — | I21413 | Life | 1:100 |
| isolectin GS-IB4 | | | (AB_2313921) | technologies | |
| conjugate | | | | | |
| Alexa Fluor 647 | — | — | I32450 | Invitrogen | |
| isolectin GS-IB4 | | | | | |
| conjugate | | | | | |
| | | | Catalog | | |
| Secondary | | | number | | |
| antibody | Type/IgG | Host | (RRID) | Company | Dilution |
| Anti-mouse Alexa | IgG(H + L) | goat | A11001 | Life | 1:500 |
| Fluor 488 | | | (AB_2534069) | Technologies | |
| Anti-mouse Alexa | IgG(H + L) | donkey | A21203 | Life | 1:500 |
| Fluor 594 | | | (AB_141633) | Technologies | |
| Anti-rat | IgG(H + L) | goat | A21247 | ThermoFisher | 1:250 |
| Alexa Fluor 647 | | | (AB 141778) | | |
| Anti-rabbit | IgG(H + L) | donkey | A21206 | Life | 1:250 |
| Alexa Fluor 488 | | | (AB_2535792) | Technologies | |
| Anti-rabbit | IgG(H + L) | goat | A11036 | ThermoFisher | 1:250 |
| Alexa Fluor 568 | | | (AB_10563566) | | |

### Example 2—A Study Evaluating Rescue Retinal Pathology in Model Norrie Disease when Codon-Optimised NDP.AAV is Administered Intravenously and Intraocularly

The Ndp-KO mouse model recapitulates human Norrie disease and Ndp-KO mice were used in experiments in which codon-optimised NDP-AAV gene therapy was administered either intravenously or intraocularly.

FIG. 16 shows the experimental design for administering codon-optimised NDP-AAV gene therapy by intravenous injection. KO mice were intravenously injected at p2 or p21 using either AAV2/9.Gfp/NDP (treatment dose) or eBSS (control dose). Genotyping was performed by PCR and the mice examined a P30 to test the retinal vascular architecture and blood retinal barrier and examined at P45 to test by electroretinogram.

FIG. 17 shows the experimental design for administering codon-optimised NDP-AAV gene therapy by intracochlear injection. KO mice were intracochlearly injected at p8 or p22 using either AAV2/ShH10.Gfp/NDP (treatment dose) or eBSS (control dose). Genotyping was performed by PCR and the mice examined a P30 to test the retinal vascular architecture and blood retinal barrier and examined at P45 to test by electroretinogram.

FIG. 18A shows a serial dilution of fluorescently labelled bovine serum albumin were made up. The fluorescence of the mixture at each concentration was measured. The relationship between BSA concentration and fluorescence was found to be linear within the dynamic range required for the subsequent experiments. Therefore, the total weight of BSA within each lysed retina can be inferred from the fluorescence reading of each. Together with BCA protein quantification, this allows a read-out of ng BSA-AF 594 per mg of retinal protein in each well.

The graph of FIG. 18B quantifies the integrity of the blood-retinal barrier in WT mice, Ndp-KO mice and Ndp-KO mice that have undergone various intravitreal treatments. BSA (bovine serum albumin) should not normally cross the blood retinal barrier. In the WT mouse a low level of fluorescently tagged BSA is therefore found in the lysed retina (shown in blue). In Ndp-KO mice a much higher amount of fluorescently tagged BSA is found in lysed retinas (shown in red). The following four green bars pertain to mice treated with 1.76×1010 vector genomes of construct 0 (SEQ ID NO 18, SEQ ID NO: 19) per eye. 3.47×109 vector genomes of construct 6 (SEQ ID NO 21, SEQ ID NO: 22) per eye, 1.74×109 vector genomes of construct 6 (SEQ ID NO 21, SEQ ID NO: 22) per eye and 8.68×108 vector genomes of construct 6 (SEQ ID NO 21, SEQ ID NO: 22) per eye. In each case the integrity of the blood-retinal barrier is rescued.

FIG. 19 shows a cross-section of the retina from an Ndp-KO mouse at p30, which had been treated with intravitreal construct 6 (SEQ ID NO 21, SEQ ID NO: 22) (3.47×109 vector genomes per eye) at p22 (Construct 6 (SEQ ID NO 21, SEQ ID NO: 22) at P22. KO). In particular, this figure shows the presence of claudin 5 within the walls of the retinal vasculature. Claudin 5 is an essential component of the blood-retinal barrier and is known to be absent in the Ndp-KO mouse. This figure therefore provides histological evidence of blood-retinal barrier rescue. Blue: DAPI, Red 1B4, Yellow: Cld 5.

As shown in FIG. 20, intravenous injection of AAV2/9.NDP at p2 and p21 restores the claudin+,PLVAP-state of adult mouse retina. This figure shows cross-sections of the retina of a WT and Ndp-KO mice at p30. Two of the Ndp-KO mice have been treated with systemic AAV2/9.NDP at p2 and at p21 respectively. In the WT mouse, PLVAP is absent and Claudin 5 is expressed. Absence of PLVAP and presence of claudin 5 are both required for the integrity of the blood retinal barrier. In the KO mouse these changes are reversed, demonstrating that the blood retinal barrier is permeable. In the P2-treated mouse the pattern seen in the WT is restored. In the P-21 treated mouse the pattern seen in the WT is partially restored (Claudin 5 is present, but PLVAP is reduced rather than absent).

Sections showing retinal vasculature—1 layer only in Ndp-KO compared to 3 in WT are shown in FIG. 21. Cross sections of the WT, Ndp-KO and Ndp-KO retina treated with AAV2/ShH10.NDP (construct 0 (SEQ ID NO 18, SEQ ID NO: 19)) are shown at P30. The treated retina was injected with an intravitreal injection at P8. In the WT, 3 intraretinal vascular layers are present. In the KO, just one is present. In the treated retina, there is restoration of the usual 3 layers of retinal vasculature.

As shown in FIG. 22, intravitreal injection of AAV2/ShH10.NDP at p8 rescues the intermediate and deep retinal capillary layers. Retinal whole mounts are displayed from mice culled at p30. The image on the left is from an eye that was treated with AAV2/ShH10.NDP (construct 0 (SEQ ID NO 18, SEQ ID NO: 19)) at p8. The image on the right is from an untreated eye. The treated retina shows a higher density of vascularisation, increased peripheral vascularisation and reduced neovascularisation. The untreated retina shows poor peripheral vascularisation, a generally reduced density of retinal vascularisation and an area of severe neovascularisation on the lower left petal (white arrow).

Intraretinal vascularisation is beneficial for retinal function whereas neovascularisation (occurring on the retinal surface in a disorganised manner) is harmful and predisposes in humans to vitreous haemorrhage and retinal detachment.

A macro was devised that measured the density of peripheral retinal vascularisation from images of retinal wholemounts (see FIG. 23). The analysis steps include 1) subtract background, 2) extract blood vessels and make image binary, 3) place a circle of set size at the centre of the retina, centred on the disk, and 4) measure the mean grey value outside of the circle (peripheral density).

FIG. 24 shows peripheral vascular density in p8 or p21 BSS-treated WT mice, BSS-treated Ndp-KO mice and AAV2/ShH10-treated Ndp-KO mice as assessed at p30. The density of peripheral retinal vascularisation was increased by intravitreal treatment with construct 0 (SEQ ID NO 18, SEQ ID NO: 19) at P8 but not at P21.

FIG. 25 shows quantification of radius of peripheral vascularisation. The analysis steps include 1) measure the total radius of the retina, 2) measure the radius of retinal vascularisation, and 3) divide the vascularised retinal radius by the total retinal radius to obtain the ratio of vascularised retinal radius to total retinal radius. The radius of peripheral retinal vascularisation was measured by dividing the radius of the maximal peripheral extent of the vasculature to the total radius of the retina.

As shown in FIG. 26, the radius of peripheral retinal vascularisation was increased by intravitreal treatment with construct 0 (SEQ ID NO 18, SEQ ID NO: 19) at P8 but not at P21.

The b wave height can be partially rescued with intravenous delivery of AAV2/9.NDP at p2 but not p21 (see FIG. 27). ERG's were performed at approximately p45. The KO Ndp-mouse exhibits a reduced b-wave amplitude, suggesting poor signal transduction from photoreceptors to bipolar cells. The mouse treated with intravenous construct 0 (SEQ ID NO 18, SEQ ID NO: 19) at p2 (early treatment) shows a statistically significant increase in the height of the b wave. However, treatment at P21 does not rescue the b wave.

As shown in FIG. 28, the b wave height can be rescued with intravenous delivery of AAV2/9.NDP (construct 0 (SEQ ID NO 18, SEQ ID NO: 19)) at p2 but not p21. The statistically significant increase in b wave height with early treatment at p2 remains up to at least 10 months of age.

### Methods Used in Example 2

Blood retinal barrier assay: To measure the trans-cellular (PLVAP—related) permeability of the blood retinal barrier, mice received a tail vein injection of fluorescently labelled bovine serum albumin (BSA-AF 594 0.5% in PBS). Three hours later the circulation was cleared with transcardial perfusion of 40 ml sterile PBS under terminal anaesthesia. During the three-hour interval between injection and perfusion, BSA-AF 594 accumulates in the retinal tissues if the blood retinal barrier is not competent. Eyes were then enucleated and either fixed in PFA in order to make flatmounts or alternatively the retina was immediately isolated. In initial experiments, retinal isolation was by the same method used for qPCR and western blot, however for later experiments the protocol was changed to careful dissection under an operating microscope in order to ensure that no iris or choroidal tissue was inadvertently included in the sample. Samples were flash frozen in 1.5 ml Eppendorf tubes on dry ice. They were later thawed on ice and lysed in RIPA buffer with cOmplete™ protease inhibitor (Roche 11836153001), spun for 5 minutes at 10,000 g and the supernatant was saved. 150 μl of supernatant per retina was divided into technical triplicates and pipetted into a black 96 well flat bottomed plate. The fluorescence of the supernatant in the red band (for BSA-AF 594) and the green band (for eGFP [enhanced green fluorescent protein] in treated animals) was measured according to the following excitor and barrier frequencies: Green; Excitation k 485 nm, Barrier k 535 nm; Red; Excitation k 580 nm, Barrier k 632 nm. The protein concentration of the samples was measured using the BCA assay according to the manufacturer's instructions in order to normalise the results to the amount of retinal protein present in each sample when calculating the results. The relationship between known BSA-AF 594 concentrations and fluorescence was also examined within the dynamic range of the experiment in order to aide interpretation of the results.

Intravitreal injections: Eyes were injected as follows: For p8 mice the lids were first divided carefully along the suture line with a scalpel, cutting from posterior to anterior whilst holding the lateral canthus up away from the eye with toothed forceps in order to avoid corneal injury. For p22 mice this was not necessary as the lids open at p12. Tropicamide 1% eye drops were applied to cause pupil dilatation, followed by proxymetacaine local anaesthetic eye drops. Viscotears were applied to prevent ocular drying and the formation of reversible cataract. A clear glass cover slip was placed over the cornea to enable direct visualisation of the posterior segment through the operating microscope. The upper and lower lids were then gently pushed apart in order to cause the eye to minimally prolapse, exposing the limbus. A 34-gauge needle on a Hamilton syringe was inserted at the limbus, angled posteriorly to avoid lens damage and 0.5μl (p8) or 1 μl (p22) of AAV2.NDP (construct 0 (SEQ ID NO 18, SEQ ID NO: 19) or 6)/ShH10 or eBSS [Earle's Balanced Salt Solution] was injected.

### Example 3—A Study Developing Gene Therapy for Norrie Disease

Norrie disease (ND) is a rare recessive X-linked disorder. As shown in FIG. 29 Norrie disease manifests as congenital blindness, followed by progressive hearing loss. It is caused by mutations in NDP gene, which encodes a secreted soluble WNT analogue norrin.

Norrie blindness is caused by failure of deep retinal vasculature to develop. Norrie patients and adult Norrie mice are known to have a cochlear vascular pathology. Schematic below shows cochlear (A) and retinal (B, C) vasculature.

A previously generated Norrie disease mouse model (Berger et al 1996) Ndp-KO recapitulated human Norrie disease. We investigated the early phenotype to identify a therapeutic window.

FIG. 31 shows retinal pathology begins from sprouting of the retinal vasculature. The deep vascular plexi fail to develop.

We established that cochlear vascular pathology also begins earlier than previously thought.

As shown in FIG. 32, as early as P10 there is a marked pathology in the spiral ligament vasculature morphology and barrier is not established (Bryant et al. 2022). Cochlear sensory hair cell degeneration follows.

Provided that the vascular pathology starts early, we conclude that the optimal rescue could be achieved with early treatment. The therapeutic window is illustrated in FIG. 33.

This poses the following hypotheses: 1) it is possible to generate a fully functional tagged NDP gene therapy construct for proof-of-concept treatment and 2) retina and cochlea of Norrie disease mice can be rescued with neonatal treatment.

### Methods Used in Example 3

1) The gene therapy construct encodes a tagged NDP to allow alternative tag-based detection and GFP to label the transduced cells (see FIG. 34).
2) The construct was tested for functionality in vitro.
3) The construct was packaged into an AAV vector, using a triple transfection protocol (see FIG. 35).
4) To establish functionality in vivo, neonatal mice were treated at the beginning of the initial vascular development in the retina and inner ear.
5) Mice were monitored for adverse effects three times a week.
6) Retinal and cochlear vascular phenotypes were analysed at 2 months of age.

### Results

In vitro tests indicate functionality of the construct.
1) The construct labelled the transduced HEK293 cells. Anti-flag tag immunostaining colocalized with the intrinsic GFP (see FIG. 36).
2) Western blot analysis detected expression of NDP protein monomer and GFP as separate proteins, as apparent from the different molecular weights of the NDP and GFP bands (see FIG. 37).
3) Norrin is a secreted signalling protein, which induces canonical WNT/0-catenin signaling through FZ4 complex with LRP5 or LRP6 and TSPAN12 (see FIG. 38A). Activation of this receptor complex inhibits the β-catenin destruction complex.

As shown in FIG. 38B, the ability of the construct to induce β-catenin signalling through these receptors was demonstrated in vitro using a TopFlash assay. Lithium was used as a positive control.

In vivo administration in Norrie mice was safe and rescued vascular morphology in the eye and ear.

As shown in FIG. 39, Ndp-KO mice, treated at neonatal age, grew and developed as normal.

Weights of treated mice were comparable to littermate controls. Retinal vascular morphology of treated Ndp-KO mice was comparable to the WT: three complete lawyers of retinal vasculature were formed. Neovascularization, as seen in the Ndp-KO mice, was also prevented by the treatment.

FIG. 40 shows the retinal vascular morphology of treated Ndp-KO mice was comparable to the WT: three complete lawyers of retinal vasculature were formed. Neovascularization, as seen in the Ndp-KO mice, was also prevented by the treatment.

FIG. 41 shows, cochlear vascular morphology of treated Ndp-KO (C) mice was comparable to the WT (A). Note the enlarged, low branching vessels in the Ndp-KO (C, arrowheads) and even distribution and comparable diameter vessels in the WT and treated Ndp-KO (C, arrows) (The scale bar represents 100 μm).

The following conclusions can be determined from this study: 1) the tagged NDP gene therapy construct produced functional NDP protein, 2) Ndp-KO mouse retinal and cochlear vascular morphology was rescued by neonatal gene therapy, and 3) This construct has potential for clinical application.

FIG. 42 shows expression of NDP constructs 0, 1, 2, and 3 in HEK293 cells using the EGFP tag.

### PUBLICATION BIBLIOGRAPHY

Apple, David J.; Fishman, Gerald A.; Goldberg, Morton F. (1974): Ocular Histopathology of Norrie's Disease. In American Journal of Ophthalmology 78 (2), pp. 196-203. DOI: 10.1016/0002-9394(74)90076-2.
Aslesh, Tejal; Yokota, Toshifumi (2022): Restoring SMN Expression: An Overview of the Therapeutic Developments for the Treatment of Spinal Muscular Atrophy. In Cells 11 (3). DOI: 10.3390/cells11030417.
Bassett, Erin A.; Tokarew, Nicholas; Allemano, Ema A.; Mazerolle, Chantal; Morin, Katy; Mears, Alan J. et al. (2016): Norrin/Frizzled4 signalling in the preneoplastic niche blocks medulloblastoma initiation. In eLife 5. DOI: 10.7554/eLife.16764.
Berger, W.; Meindl, A.; van de Pol, T. J.; Cremers, F. P.; Ropers, H. H.; Doerner, C. et al. (1992a): Isolation of a candidate gene for Norrie disease by positional cloning. In Nature genetics 1 (3), pp. 199-203. DOI: 10.1038/ng0692-199.
Berger, W.; van de Pol, D.; Bachner, D.; Oerlemans, F.; Winkens, H.; Hameister, H. et al. (1996): An animal model for Norrie disease (ND): gene targeting of the mouse ND gene. In Human molecular genetics 5 (1), pp. 51-59. DOI: 10.1093/hmg/5.1.51.
Berger, W.; van de Pol, D.; Warburg, M.; Gal, A.; Bleeker-Wagemakers, L.; Silva, H. de et al. (1992b): Mutations in the candidate gene for Norrie disease. In Human molecular genetics 1 (7), pp. 461-465. DOI: 10.1093/hmg/1.7.461.
Bryant, Dale; Pauzuolyte, Valda; Ingham, Neil J.; Patel, Aara; Pagarkar, Waheeda; Anderson, Lucy A. et al. (2022): The timing of auditory sensory deficits in Norrie disease has implications for therapeutic intervention. In JCI insight 7 (3). DOI: 10.1172/jci.insight.148586.
Cagao, Gongalo; Garrido, Cristina; Miranda, Vasco; Pinto-Basto, Jorge; Chaves, Joao; Chorao, Rui (2018): Refractory epilepsy in Norrie disease. In Neurological sciences: official journal of the Italian Neurological Society and of the Italian Society of Clinical Neurophysiology 39 (9), pp. 1631-1633. DOI: 10.1007/s10072-018-3428-9.
Chang, Tao-Hsin; Hsieh, Fu-Lien; Zebisch, Matthias; Harlos, Karl; Elegheert, Jonathan; Jones, E. Yvonne (2015): Structure and functional properties of Norrin mimic Wnt for signalling with Frizzled4, Lrp5/6, and proteoglycan. In eLife 4. DOI: 10.7554/eLife.06554.
Chen, Z. Y.; Hendriks, R. W.; Jobling, M. A.; Powell, J. F.; Breakefield, X. O.; Sims, K. B.; Craig, I. W. (1992): Isolation and characterization of a candidate gene for Norrie disease. In Nature genetics 1 (3), pp. 204-208. DOI: 10.1038/ng0692-204.
Chin-Ju, Hu; Ying-Chang, Lu; Yi-Hsiu, Tsai; Haw-Yuan, Cheng; Hiroki, Takeda; Chun-Ying, Huang; Ru, Xiao; Chuan-Jen, Hsu; Jin-Wu, Tsai: Luk, H. Vandenberghe; Chen-Chi, Wu; Yen-Fu, Cheng (2020): Efficient In Utero Gene Transfer to the Mammalian Inner Ears by the Synthetic Adeno-Associated Viral Vector Anc80L65. In Molecular Therapy 18(12) pp 493-500.
Drenser, Kimberly A.; Fecko, Alice; Dailey, Wendy; Trese, Michael T. (2007): A characteristic phenotypic retinal appearance in Norrie disease. In Retina (Philadelphia, Pa.) 27 (2), pp. 243-246. DOI: 10.1097/01.iae.0000231380.29644.c3.
Eilken, Hanna M.; Dieguez-Hurtado, Rodrigo; Schmidt, Inga; Nakayama, Masanori; Jeong, Hyun-Woo; Arf, Hendrik et al. (2017): Pericytes regulate VEGF-induced endothelial sprouting through VEGFR1. In Nature communications 8 (1), p. 1574. DOI: 10.1038/s41467017-01738-3.
Fradkin, Allan H. (1971): Norrie's Disease Congenital Progressive Oculo-Acoustico-Cerebral Degeneration. In American Journal of Ophthalmology 72 (5), pp. 947-948. DOI: 10.1016/0002-9394(71)91694-1.
Fruttiger, Marcus (2002): Development of the mouse retinal vasculature: angiogenesis versus vasculogenesis. In Investigative ophthalmology & visual science 43 (2), pp. 522-527.
Hayashi, Yushi; Chiang, Hao; Tian, ChunJie; Indzhykulian, Artur A.; Edge, Albert S. B. (2021): Norrie disease protein is essential for cochlear hair cell maturation. In Proceedings of the National Academy of Sciences of the United States of America 118 (39). DOI: 10.1073/pnas.2106369118.
Holmes, Lewis B. (1971): Norrie's disease. An X-linked syndrome of retinal malformation, mental retardation, and deafness. In The Journal of Pediatrics 79 (1), pp. 89-92. DOI: 10.1016/50022-3476(71)80063-X.
Johnson, K. R.; Erway, L. C.; Cook, S. A.; Willott, J. F.; Zheng, Q. Y. (1997): A major gene affecting age-related hearing loss in C57BL/6J mice. In Hear Res. 114(1-2), pp 83-92. DOI: 10.1016/s0378-5955(97)00155-x.
Junge, Harald J.; Yang, Stacey; Burton, Jeremy B.; Paes, Kim; Shu, Xiao; French, Dorothy M. et al. (2009): TSPAN12 regulates retinal vascular development by promoting Norrin—but not Wnt-induced FZD4/beta-catenin signaling. In Cell 139 (2), pp. 299-311. DOI: 10.1016/j.cell.2009.07.048.
Li, Meng; Mei, Lingyun; He, Chufeng; Chen, Hongsheng; Cai, Xinzhang; Liu, Yalan et al. (2019): Extrusion pump ABCC1 was first linked with nonsyndromic hearing loss in humans by stepwise genetic analysis. In Genetics in medicine: official journal of the American College of Medical Genetics 21 (12), pp. 2744-2754. DOI: 10.1038/s41436-019-0594-y.
Liu, Huizhan; Li, Yi; Chen, Lei; Zhang, Qian; Pan, Ning; Nichols, David H. et al. (2016): Organ of Corti and Stria Vascularis: Is there an Interdependence for Survival? In PloS one 11 (12), e0168953. DOI: 10.1371/journal.pone.0168953.
Luhmann, Ulrich F. O.; Lin, Jihong; Acar, Niyazi; Lammel, Stefanie; Feil, Silke; Grimm, Christian et al. (2005a): Role of the Norrie disease pseudoglioma gene in sprouting angiogenesis during development of the retinal vasculature. In Investigative ophthalmology & visual science 46 (9), pp. 3372-3382. DOI: 10.1167/iovs.05-0174.
Luhmann, Ulrich F. O.; Meunier, Dominique; Shi, Wei; Littges, Angela; Pfarrer, Christiane; Fundele, Reinald; Berger, Wolfgang (2005b): Fetal loss in homozygous mutant Norrie disease mice: a new role of Norrin in reproduction. In Genesis (New York, N.Y.: 2000) 42 (4), pp. 253-262. DOI: 10.1002/gene.20141.
Massaro, Giulia; Hughes, Michael P.; Whaler, Sammie M.; Wallom, Kerri-Lee; Priestman, David A.; Platt, Frances M. et al. (2020): Systemic AAV9 gene therapy using the synapsin I promoter rescues a mouse model of neuronopathic Gaucher disease but with limited cross-correction potential to astrocytes. In Human molecular genetics 29 (12), pp. 1933-1949. DOI: 10.1093/hmg/ddz317.
Merkel, Steven F.; Andrews, Allison M.; Lutton, Evan M.; Mu, Dakai; Hudry, Eloise; Hyman, Bradley T. et al. (2017): Trafficking of adeno-associated virus vectors across a model of the blood-brain barrier; a comparative study of transcytosis and transduction using primary human brain endothelial cells. In Journal of neurochemistry 140 (2), pp. 216-230. DOI: 10.1111/jnc.13861.
Michaelides, M.; Luthert, P. J.; Cooling, R.; Firth, H.; Moore, A. T. (2004): Norrie disease and peripheral venous insufficiency. In The Britishjournal of ophthalmology 88 (11), p. 1475. DOI: 10.1136/bjo.2004.042556.
Nadol, J. B.; Eavey, R. D.; Liberfarb, R. M.; Merchant, S. N.; Williams, R.; Climenhager, D.; Albert, D. M. (1990): Histopathology of the ears, eyes, and brain in Norrie's disease (oculoacousticocerebral degeneration). In American journal of otolaryngology 11 (2), pp. 112-124.
Ohlmann, Andreas; Scholz, Michael; Goldwich, Andreas; Chauhan, Bharesh K.; Hudl, Kristiane; Ohlmann, Anne V. et al. (2005): Ectopic norrin induces growth of ocular capillaries and restores normal retinal angiogenesis in Norrie disease mutant mice. In The Journal of neuroscience: the official journal of the Society for Neuroscience 25 (7), pp. 1701-1710. DOI: 10.1523/JNEUROSCI.4756-04.2005.
Ohlmann, Andreas; Tamm, Ernst R. (2012): Norrin: molecular and functional properties of an angiogenic and neuroprotective growth factor. In Progress in retinal and eye research 31 (3), pp. 243-257. DOI: 10.1016/j.preteyeres.2012.02.002.
Parving, A.; Elberling, C.; Warburg, M. (1978): Electrophysiological study of Norrie's disease. An X-linked recessive trait with hearing loss. In Audiology: official organ of the International Society of Audiology 17 (4), pp. 293-298.
Pronobis, Mira I.; Deuitch, Natalie; Peifer, Mark (2016): The Miraprep: A Protocol that Uses a Miniprep Kit and Provides Maxiprep Yields. In PloS one 11 (8), e0160509. DOI: 10.1371/journal.pone.0160509.
Redmond, R. M.; Vaughan, J. I.; Jay, M.; Jay, B. (1993): In-utero diagnosis of Norrie disease by ultrasonography. In Ophthalmic paediatrics and genetics 14 (1), pp. 1-3.
Rehm, Heidi L.; Gutierrez-Espeleta, Gustavo A.; Garcia, Rafael; Jimenez, Gerardo; Khetarpal, Umang; Priest, Janice M. et al. (1997): Norrie disease gene mutation in a large Costa Rican kindred with a novel phenotype including venous insufficiency. In Hum. Mutat. 9 (5), pp. 402-408. DOI: 10.1002/(SICI)1098-1004(1997)9:5<402::AID-HUMU4>3.0.CO;2-5.
Rehm, Heidi L.; Zhang, Duan-Sun; Brown, M. Christian; Burgess, Barbara; Halpin, Chris; Berger, Wolfgang et al. (2002): Vascular defects and sensorineural deafness in a mouse model of Norrie disease. In The Journal of neuroscience: the official journal of the Society for Neuroscience 22 (11), pp. 4286-4292.
Richter, M.; Gottanka, J.; May, C. A.; Welge-Lissen, U.; Berger, W.; Litjen-Drecoll, E. (1998): Retinal vasculature changes in Norrie disease mice. In Investigative ophthalmology & visual science 39 (12), pp. 2450-2457.
Shibata, Seiji B.; Yoshimura, Hidekane; Ranum, Paul T.; Goodwin, Alexander T.; Smith, Richard J. H. (2017): Intravenous rAAV2/9 injection for murine cochlear gene delivery. In Scientific reports 7 (1), p. 9609. DOI: 10.1038/s41598-017-09805-x.
Sisk, Robert A.; Hufnagel, Robert B.; Bandi, Sindura; Polzin, William J.; Ahmed, Zubair M. (2014): Planned preterm delivery and treatment of retinal neovascularization in Norrie disease. In Ophthalmology 121 (6), pp. 1312-1313. DOI: 10.1016/j.ophtha.2014.01.001.
Smith, Sharon E.; Mullen, Thomas E.; Graham, Dionne; Sims, Katherine B.; Rehm, Heidi L. (2012): Norrie disease: extraocular clinical manifestations in 56 patients. In American journal of medical genetics. Part A 158A (8), pp. 1909-1917. DOI: 10.1002/ajmg.a.35469.
Verdoodt, Dorien; Peeleman, Noa; van Camp, Guy; van Rompaey, Vincent; Ponsaerts, Peter (2021): Transduction Efficiency and Immunogenicity of Viral Vectors for Cochlear Gene Therapy: A Systematic Review of Preclinical Animal Studies. In Frontiers in cellular neuroscience 15, p. 728610. DOI: 10.3389/fncel.2021.728610.
Vikhe Patil, Kim; Canlon, Barbara; Cederroth, Christopher R. (2015): High quality RNA extraction of the mammalian cochlea for qRT-PCR and transcriptome analyses. In Hearing research 325, pp. 42-48. DOI: 10.1016/j.heares.2015.03.008.
Wang, Xiaohan; Zhang, Jinhui; Li, Guangshuai; Sai, Na; Han, Jiang; Hou, Zhiqiang et al. (2019): Vascular regeneration in adult mouse cochlea stimulated by VEGF-A165 and driven by NG2-derived cells ex vivo. In Hearing research 377, pp. 179-188. DOI: 10.1016/j.heares.2019.03.010.
Wang, Yanshu; Rattner, Amir; Zhou, Yulian; Williams, John; Smallwood, Philip M.; Nathans, Jeremy (2012): Norrin/Frizzled4 signaling in retinal vascular development and blood brain barrier plasticity. In Cell 151 (6), pp. 1332-1344. DOI: 10.1016/j.cell.2012.10.042.
Wawrzynski, James; Patel, Aara; Badran, Abdul; Dowell, Isaac; Henderson, Robert; Sowden, Jane. C (2022): Spectrum of Mutations in NDP Resulting in Ocular Disease; a Systematic Review. In Front Genet. 16 (13):884722. doi: 10.3389/fgene.2022.884722.
Xu, Qiang; Wang, Yanshu; Dabdoub, Alain; Smallwood, Philip M.; Williams, John; Woods, Chad et al. (2004): Vascular development in the retina and inner ear. Control by Norrin and Frizzled-4, a high-affinity ligand-receptor pair. In Cell 116 (6), pp. 883-895.
Yahyaoui, Raquel; Perez-Frias, Javier (2019): Amino Acid Transport Defects in Human Inherited Metabolic Disorders. In International journal of molecular sciences 21 (1). DOI: 10.3390/ijms21010119.
Ye, Xin; Smallwood, Philip; Nathans, Jeremy (2011): Expression of the Norrie disease gene (Ndp) in developing and adult mouse eye, ear, and brain. In Gene expression patterns: GEP 11 (1-2), pp. 151-155. DOI: 10.1016/j.gep.2010.10.007.
Ye, Xin; Wang, Yanshu; Cahill, Hugh; Yu, Minzhong; Badea, Tudor C.; Smallwood, Philip M. et al. (2009): Norrin, frizzled-4, and Lrp5 signaling in endothelial cells controls a genetic program for retinal vascularization. In Cell 139 (2), pp. 285-298. DOI: 10.1016/j.cell.2009.07.047.
Ysuda, Masahiro; Nagappan-Chettair, Sivapratha; Umemori, Hisashi (2021): In utero intraocular AAV injection for early gene expression in the developing rodent retina. In STAR Protocols 2(3), pp 1-11
Zhang, Jinhui; Hou, Zhiqiang; Wang, Xiaohan; Jiang, Han; Neng, Lingling; Zhang, Yunpei et al. (2021): VEGFA165 gene therapy ameliorates blood-labyrinth barrier breakdown and hearing loss. In JCI insight 6 (8). DOI: 10.1172/jci.insight.143285.

1. A construct comprising a wildtype NDP nucleic acid sequence or a codon-optimised NDP nucleic acid sequence.

2. (canceled)

3. The construct according to claim 1, wherein the wildtype NDP nucleic acid sequence or the codon-optimised NDP nucleic acid sequence comprises SEQ ID NO: 1, SEQ ID NO: 3, or a nucleotide sequence having at least about 90%, 91%, 92%, 93%, 94%, 95%, 96%, 97%, 98%, or 99% sequence identity to SEQ ID NO: 1 or SEQ ID NO: 3.

4-6. (canceled)

7. The construct according to claim 1, wherein the wildtype NDP nucleic acid sequence or the codon-optimised NDP nucleic acid sequence is under the control of a CAG promoter or a CBA promoter.

8-9. (canceled)

10. The construct according to claim 1, wherein the construct further comprises one or more of the following elements: a WPRE element, 5′ and 3′ inverted terminal repeats, a self-cleaving P2A linker, a FLAG epitope sequence tagging C terminus, a simian virus 40 PolyA (SV40 late polyA) sequence, bovine growth hormone polyadenylation signal (BGHpA), an EGFP open reading frame sequence, and a pUC ori.

11. The construct according to claim 1, wherein the wildtype NDP nucleic acid sequence or the codon-optimised NDP nucleic acid sequence is incorporated into one or more of the following vectors: AAV1, AAV2, AAV3, AAV4, AAV5, AAV6, AAV7, AAV8, AAV9, AAV10, AAV-rh8, AAV-rh10, AAV-rh39, AAV-rh43, AAVAnc80, AAV 2/ShH10, AAV 2/9 vector, or AAV-S vector.

12-14. (canceled)

15. The construct according to claim 1, wherein the construct is formulated for at least one of intravenous administration, intraocular administration, or intracochlear administration.

16-18. (canceled)

19. A method of treating a disease, the method comprising administering a therapeutically effective amount of a construct comprising wildtype NDP nucleic acid sequence or a codon-optimised NDP nucleic acid sequence to a subject in need thereof, wherein the disease is selected from the group consisting of Norrie disease, age related hearing loss, diabetic maculopathy and retinopathy, retinal neovascularisation, retinal exudation, retinopathy of prematurity (ROP), Familial exudative vitreoretinopathy (FEVR), Coats disease, and other NDP-related diseases, and other beta catenin signalling-related retinal diseases.

20. The method of claim 19, wherein an AAV particle (NDP.AAV) comprises the construct.

21. (canceled)

22. The method according to claim 20, wherein the AAV particle is administered intravenously, intraocularly, or intracochlearly.

23. The method according to claim 22, wherein the AAV particle is an AAV 2/9 particle or an AAV 2/ShH10 particle.

24-27. (canceled)

28. The method according to claim 20, wherein a first dose is administered intraocularly neonatally.

29. The method according to claim 28, wherein the first dose is administered at about 14-17 postconceptual weeks (pcws).

30. The method according to claim 28, wherein a second dose is administered intraocularly neonatally or at any time after birth.

31. The method according to claim 30, wherein the second dose is administered at about 22-26 postconceptual weeks (pcws).

32. (canceled)

33. The method according to claim 30, wherein a third dose is administered intraocularly at any time after birth.

34. The method according to claim 20, wherein a first dose is administered intracochlearly neonatally.

35. The method according to claim 28, wherein the first dose is administered at about 13-20 postconceptual weeks (pcws).

36. The method according to claim 28, wherein a second dose is administered intracochlearly to an adolescent or in adulthood.

37. (canceled)

38. The method according to claim 36, wherein a third dose is administered intracochlearly in adulthood, more preferably ages 12 years and above.

39. The method according to claim 20, wherein a first dose is administered intravenously.

40-42. (canceled)

43. The method according to claim 20, wherein the AAV particle is adapted to target fibrocytes of spiral ligament (SL), stria vascularis and lateral wall basal cell and marginal cells, glial cells of the modiolus; evidence for rescue after transduction of retinal muller cells, RGCs, RPE cells and PRS, RGC alone and/or Muller cells alone.

44. A method of rescuing vascular architecture or barrier function in the ear and the eye, the method comprising the step of administering an NDP.AAV particle comprising the construct of claim 1 by at least one of intravenous administration, intraocular administration, and intracochlear administration at an early stage of development, preferably a pre-natal stage of development, or alternatively in the first year of life, preferably in the neonatal period.

45. (canceled)

46. A method of preventing or treating neovascularisation in the eye, the method comprising the step of administering an NDP.AAV particle comprising the construct of claim 1 by at least one of intravenous administration or intraocular administration at an early stage of development or at a late stage of development.

47. A method of rescuing the blood retinal barrier and/or barrier in the cochlear and/or the endocochlear potential, the method comprising the step of administering an NDP.AAV particle comprising the construct of claim 1 by at least one of intravenous administration, intraocular administration, and intracochlear administration at a late stage of development, preferably a post-natal stage of development.

48. (canceled)


SEQUENCE LISTING (24 sequences)

SEQ ID NO: 1 - DNA, 402 bp, Homo sapiens
  source 1..402: mol_type = genomic DNA
atgagaaaacatgtactagctgcatccttttctatgctctccctgctggtgataatgggagatacagacagtaaaacggacagctcattcataatggactcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctcagggggcatgcgactcactgccacctaccggtacatcctctcctgtcactgcgaggaatgcaattcctga

SEQ ID NO: 2 - protein, 133 aa, Homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 3 - DNA, 402 bp, Homo sapiens
  source 1..402: mol_type = genomic DNA
atgcgcaagcacgtcctcgcagcttcattctctatgttgtcactcctggtgataatgggcgacacggacagcaaaacggacagcagttttatcatggactccgacccgagaagatgtatgcgccatcactatgttgatagtatctcccatcccctgtataagtgttcttcaaaaatggtcctcctcgctcgctgcgaaggccactgttctcaagcctcccgctccgagccgctggtctcatttagcactgtcttgaaacaaccctttcgctccagctgccactgctgccggcctcaaacgtcaaaactcaaagcactgcgactgcgatgctctggggggatgcggctcactgcgacctaccgatacattctttcctgtcattgcgaggaatgtaattcttga

SEQ ID NO: 4 - protein, 133 aa, Homo sapiens; identical to SEQ ID NO: 2

SEQ ID NO: 5 - DNA, 23 bp, synthetic construct
  source 1..23: mol_type = other DNA
ttaaggcacgggtagcactcacg

SEQ ID NO: 6 - DNA, 23 bp, synthetic construct
  source 1..23: mol_type = other DNA
ttagacatagttcttcttgtcgt

SEQ ID NO: 7 - DNA, 20 bp, synthetic construct
  source 1..20: mol_type = other DNA
gtggttggactatctgcctc

SEQ ID NO: 8 - DNA, 18 bp, synthetic construct
  source 1..18: mol_type = other DNA
atagcggcgatgaagcga

SEQ ID NO: 9 - DNA, 20 bp, synthetic construct
  source 1..20: mol_type = other DNA
tgttaccaactgggacgaca

SEQ ID NO: 10 - DNA, 19 bp, synthetic construct
  source 1..19: mol_type = other DNA
ctgggtcatctttcacggt

SEQ ID NO: 11 - DNA, 21 bp, synthetic construct
  source 1..21: mol_type = other DNA
ggaacccctagtgatggagtt

SEQ ID NO: 12 - DNA, 16 bp, synthetic construct
  source 1..16: mol_type = other DNA
cggcctcagtgagcga

SEQ ID NO: 13 - DNA, 19 bp, synthetic construct
  source 1..19: mol_type = other DNA
agtccgccctgagcaaaga

SEQ ID NO: 14 - DNA, 20 bp, synthetic construct
  source 1..20: mol_type = other DNA
tccagcaggaccatgtgatc

SEQ ID NO: 15 - DNA, 22 bp, synthetic construct
  source 1..22: mol_type = other DNA
gtattgcatccatatttcttgg

SEQ ID NO: 16 - DNA, 21 bp, synthetic construct
  source 1..21: mol_type = other DNA
ctctccatcccctgacaagga

SEQ ID NO: 17 - DNA, 4401 bp, synthetic construct
  source 1..4401: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatgagaaaacatgtactagctgcatccttttctatgctctccctgctggtgataatgggagatacagacagtaaaacggacagctcattcataatggactcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctcagggggcatgcgactcactgccacctaccggtacatcctctcctgtcactgcgaggaatgcaattccgactacaaagacgatgacgacaagtgaacccagctttcttgtacaaagtggtgatggccggcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 7282 bp, synthetic construct
  source 1..7282: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatgagaaaacatgtactagctgcatccttttctatgctctccctgctggtgataatgggagatacagacagtaaaacggacagctcattcataatggactcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctcagggggcatgcgactcactgccacctaccggtacatcctctcctgtcactgcgaggaatgcaattccgactacaaagacgatgacgacaagtgaacccagctttcttgtacaaagtggtgatggccggcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 19 - DNA, 4161 bp, synthetic construct
  source 1..4161: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatgcgcaagcacgtcctcgcagcttcattctctatgttgtcactcctggtgataatgggcgacacggacagcaaaacggacagcagttttatcatggactccgacccgagaagatgtatgcgccatcactatgttgatagtatctcccatcccctgtataagtgttcttcaaaaatggtcctcctcgctcgctgcgaaggccactgttctcaagcctcccgctccgagccgctggtctcatttagcactgtcttgaaacaaccctttcgctccagctgccactgctgccggcctcaaacgtcaaaactcaaagcactgcgactgcgatgctctggggggatgcggctcactgcgacctaccgatacattctttcctgtcattgcgaggaatgtaattctgactacaaagacgatgacgacaaggactacaaagacgatgacgacaagtgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 6758 bp, synthetic construct
  source 1..6758: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatgcgcaagcacgtcctcgcagcttcattctctatgttgtcactcctggtgataatgggcgacacggacagcaaaacggacagcagttttatcatggactccgacccgagaagatgtatgcgccatcactatgttgatagtatctcccatcccctgtataagtgttcttcaaaaatggtcctcctcgctcgctgcgaaggccactgttctcaagcctcccgctccgagccgctggtctcatttagcactgtcttgaaacaaccctttcgctccagctgccactgctgccggcctcaaacgtcaaaactcaaagcactgcgactgcgatgctctggggggatgcggctcactgcgacctaccgatacattctttcctgtcattgcgaggaatgtaattctgactacaaagacgatgacgacaaggactacaaagacgatgacgacaagtgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 21 - DNA, 3330 bp, synthetic construct
  source 1..3330: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgcgcaagcacgtcctcgcagcttcattctctatgttgtcactcctggtgataatgggcgacacggacagcaaaacggacagcagttttatcatggactccgacccgagaagatgtatgcgccatcactatgttgatagtatctcccatcccctgtataagtgttcttcaaaaatggtcctcctcgctcgctgcgaaggccactgttctcaagcctcccgctccgagccgctggtctcatttagcactgtcttgaaacaaccctttcgctccagctgccactgctgccggcctcaaacgtcaaaactcaaagcactgcgactgcgatgctctggggggatgcggctcactgcgacctaccgatacattctttcctgtcattgcgaggaatgtaattcttgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 22 - DNA, 5927 bp, synthetic construct
  source 1..5927: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgcgcaagcacgtcctcgcagcttcattctctatgttgtcactcctggtgataatgggcgacacggacagcaaaacggacagcagttttatcatggactccgacccgagaagatgtatgcgccatcactatgttgatagtatctcccatcccctgtataagtgttcttcaaaaatggtcctcctcgctcgctgcgaaggccactgttctcaagcctcccgctccgagccgctggtctcatttagcactgtcttgaaacaaccctttcgctccagctgccactgctgccggcctcaaacgtcaaaactcaaagcactgcgactgcgatgctctggggggatgcggctcactgcgacctaccgatacattctttcctgtcattgcgaggaatgtaattcttgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 23 - DNA, 3928 bp, synthetic construct
  source 1..3928: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggattctgtttgtgactggctggtccaaggtagacagggtatttttttttccaagtggagcataagaaaagggatttctggcaataaaactgaaagttttaagacacctcttgcaaatatcagtgactggagtaaaggtcccatggaaaagtaataaggtgaatgtaaatttattaatgaataacaaatagtttgatacagagttgaacaagacagtttcagcatcaacaattacatgaagctcccaagaaaaatttctttttgagttttataatgtattattttttccacgaaatatatatattttaaattctcagtttaattcttcatcagtagtattaataaaacaaattattctgggtctgatttacctgacataaaataattattaaatgttctttgtattttcctccaaaatccctgacggttttctgtgttaattttaagctttcaaagggcaagaattatgaaatatttgcctttgtagtcccacagctcataacacagcatgattcagctgcaggtatttaacccacacacatagttacctttgtcacttttctactagttgttgtgtaatgggttagctttatctattagtttctccttcatatagctcaaggagtcaaggaatacacgttctcattgtttctaaatcagacctaaagttttattctaaatgatgcagatgaaggggcttattaaagtgccattgtgaatttaatcatgtatatgctgctaaaaatcatttaatggatgaacaacccggaaaaaaagaactctcaccacccacacacatcgtgataaaatagggcagcgttttgcacttgctttaacagcatcgcctgagaaaaaaatttctggttcccatctttcccctctcttacttaaaatttcaacttcatcacagtcagctgccgaatcgttcaacagaatgccacactgcctttgtatttccaaaactatacgtctctattgcggacggcacatctttatggcagccacatgcttgaaaaagaattaaattcagaatattcatttggcctcttattagttccataataccattaaaaaagaaagaaagaaagaaacttcctcgcccttgttctgctacgctgttcccatcgtaagatgctccgtggaagggagccgagcggtgggcagaggctgagtccccgataacgagcgcctcacatttccgtggcattcccatttgctagtgcgctgctgcggccgcacgcctgattgatatatgactgcaatggcacttttccatttgacattctctctctctctctccctctctctctctccctctctctctccctctctctctctccctgtgtcgcttaaacaacagtcctaacttttgtgtgttgcaaatataaaaggcaagccatgtgacagagggacagaagaacaaaagcatttggaagtaacaggacctctttctagctctcagaaaagtctgagaagaaaggagccctgcgttcccctaagcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatgcgcaagcacgtcctcgcagcttcattctctatgttgtcactcctggtgataatgggcgacacggacagcaaaacggacagcagttttatcatggactccgacccgagaagatgtatgcgccatcactatgttgatagtatctcccatcccctgtataagtgttcttcaaaaatggtcctcctcgctcgctgcgaaggccactgttctcaagcctcccgctccgagccgctggtctcatttagcactgtcttgaaacaaccctttcgctccagctgccactgctgccggcctcaaacgtcaaaactcaaagcactgcgactgcgatgctctggggggatgcggctcactgcgacctaccgatacattctttcctgtcattgcgaggaatgtaattctgactacaaagacgatgacgacaaggactacaaagacgatgacgacaagtgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 4187 bp, synthetic construct
  source 1..4187: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctcctcttcccatgacggccagtgactgcttctttgcccaggccctctgcactaggccagcagccagtgggagcagaggggatgggaaggcaggtggacagactgtgaagtgctaaggcagagcggggaagcagggctgtgagacagggtgggtgagctccagggcctgggttgctgtggcctgccctggctgtgatcagctgtcatttcctctttgtgtatgacactgtggaacatattaaaatttctctgggatggtatgtttggaattcctctgatgccccacctcaccaacttacccggacaataaaagcaaggttgtaagcctttgaacagctggctggctatatatttcccttctttctttcgcataatctaattcagaaaagaatccagctagactggcagtgccatgtctgggtacttgtttcctgcaagtccttcctcctccacctcctcccgccagtcctggaatcttcctgggtggcatgtggcttttcttttcttttgctcaccactgctcccagcccgcccccttttcccttcgcctgagctccttcctttgtcagctggctctttgctctgcctgttttcccatggagcccagtatcttccccagcaccctcatcccacttacaatgggggcactattaacgcaggcaagactgcgagtttaaaagcgcactgtttacttttattactacttatgtatgctgctgatctcctgccagggctctgccggagtgctcagaatgacttcaaacaataggcaacatttatttctagctttctgtgtcacgctttgcaggaatcctggccagaaaatgtattcctgactgtttccatggctcttactcagctacaagtcatttcccctttcagattatttccagttttaaggcccaatttccatttgctttttgatcttctgcttttaagatatttagccaatatcatcatcaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatgcgcaagcacgtcctcgcagcttcattctctatgttgtcactcctggtgataatgggcgacacggacagcaaaacggacagcagttttatcatggactccgacccgagaagatgtatgcgccatcactatgttgatagtatctcccatcccctgtataagtgttcttcaaaaatggtcctcctcgctcgctgcgaaggccactgttctcaagcctcccgctccgagccgctggtctcatttagcactgtcttgaaacaaccctttcgctccagctgccactgctgccggcctcaaacgtcaaaactcaaagcactgcgactgcgatgctctggggggatgcggctcactgcgacctaccgatacattctttcctgtcattgcgaggaatgtaattctgactacaaagacgatgacgacaaggactacaaagacgatgacgacaagtgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg